(19) United States
(12) Patent Application Publication
(10) Pub. No.: US 2023/0326617 A1
(43) Pub. Date: Oct. 12, 2023

(54) SYSTEM AND METHOD FOR OPTIMIZING ENERGY TRANSFER AND CONVERSION IN QUANTUM SYSTEMS

(71) Applicant: FLORIAN METZLER, CAMBRIDGE, MA (US)

(72) Inventor: FLORIAN METZLER, CAMBRIDGE, MA (US)

(73) Assignee: CAMBRIDGE PHONON SYSTEMS, INC, CAMBRIDGE, MA (US)

(21) Appl. No.: 18/022,157

(22) PCT Filed: Aug. 23, 2021
(86) PCT No.: PCT/US2021/047098

§ 371 Date: Feb. 19, 2023

Related U.S. Application Data
(63) Continuation of application No. 15/733,950, filed on Dec. 1, 2020.
(60) Provisional application No. 63/069,146, filed on Aug. 23, 2020.
(60) Provisional application No. 63/157,800, filed on Mar. 7, 2021.
(60) Provisional application No. 63/186,240, filed on May 10, 2021.
(60) Provisional application No. 63/186,249, filed on May 10, 2021.
(60) Provisional application No. 62/679,974, filed on Jun. 3, 2018.
(60) Provisional application No. 62/680,579, filed on Jun. 4, 2018.
(60) Provisional application No. 62/681,088, filed on Jun. 5, 2018.
(60) Provisional application No. 62/806,071, filed on Feb. 15, 2019.
(60) Provisional application No. 62/822,790, filed on Mar. 23, 2019.
(60) Provisional application No. 62/822,970, filed on Mar. 24, 2019.

(51) Int. Cl. G21B 1/19; G21B 1/00; G21B 3/00

(52) U.S. Cl. CPC G21B 1/19; G21B 1/00; G21B 3/006

(57) ABSTRACT

A computer implemented method for optimizing energy transfer and conversion in quantum systems and conversion in quantum systems including providing a database of input variables, modeling an intial crystal structure of the lattice sample at a first set of environmental parameters, adding a dopant and determining a new equilibrium state of the lattice sample at a second set of environmental parameters, estimating state transition rates in the absence of any strong coupling to a second quantum system, determining presence of any coupling and coupling strength to the second quantum system, providing coherent stimulation of the lattice sample, determining presence of coupling and enhancement of coupling strength after coherent stimulation, determining energy transfer and conversion dynamics as a result of coherent stimulation and the enhanced coupling and determining output variable of the energy transfer and conversion dynamics between the first quantum system and the second quantum system in the lattice sample via a computing engine.

### CROSS REFERENCE TO RELATED CO-PENDING APPLICATIONS

This application claims the benefit of U.S. provisional application Ser. No. 63/069,146 filed Aug. 23, 2020 and entitled “System and method for applications of nuclear excitation transfer”, the contents of which are expressly incorporated herein by reference.

This application also claims the benefit of U.S. provisional application Ser. No. 63/157,800 filed Mar. 7, 2021 and entitled “The case for quantum-coherent nuclear engineering”, the contents of which are expressly incorporated herein by reference.

This application claims the benefit of U.S. provisional application Ser. No. 63/186,240 filed May 10, 2021 and entitled “Process for modeling or designing systems with nuclear reaction rate enhancement or nuclear reaction product changes”, the contents of which are expressly incorporated herein by reference.

This application claims the benefit of U.S. provisional application Ser. No. 63/186,249 filed May 10, 2021 and entitled “Method and system for enhancing nuclear transition rates and modifying nuclear reaction products in condensed matter environments”, the contents of which are expressly incorporated herein by reference.

This application is a continuation-in-part of and claims the benefit of U.S. nonprovisional application Ser. No. 15/733,950 filed Dec. 1, 2020 and entitled “System and method for phonon-mediated excitation and de-excitation of nuclear states”, the contents of which are expressly incorporated herein by reference.

### FIELD OF THE INVENTION

The present invention relates to a system and method for optimizing energy transfer and conversion in quantum systems and in particular to a system and method for optimizing energy transfer and conversion in quantum systems by using quantum simulations.

### BACKGROUND OF THE INVENTION

Quantum systems, such as atoms, molecules, nuclei, can be in an energetic ground state or in an energetic excited state. Excited states are characterized by the amount of energy held by the quantum system in the respective state (compared to the ground state) and by the lifetime of the state. When a quantum system is in an excited state, it can become de-excited by turning to lower excited states or to an unexcited state of the quantum system. In the course of the de-excitation, energy of an amount corresponding to the difference between the original state and the resulting state transfers out of the quantum system. Depending on the quantum system and state, the lifetime of a state can range from less than nanoseconds to more than millions of years.

For both experiments and technical applications of energy transfer in quantum systems, it is desirable to achieve large transition rates for state transitions of interest, and also to achieve deliberate control over the final products that result from the dynamics of the engineered systems. This way, observable — and in many cases useful — outcomes can be achieved as a result of designing, assembling, and stimulating coupled quantum systems. This pertains particularly to enhanced state transitions that ultimately result in large amounts of emitted energy and/or the production of energetic particles and, more generally speaking, to enhanced state transitions that result in the accelerated conversion of potential energy to kinetic energy. However, to date, experimental research concerned with quantum coherent enhancement of state transitions, particularly in the nuclear domain, has been dominated by Edisonian trial and error approaches where researchers repeatedly adjust experimental variables in the hope of improved performance (whereas performance here refers to the values of output variables of interest such as state transition rates). An Edisoninan trial and error approach can occasionally lead to improved performance such as further enhancement of transition rates. However, such stumbled-across performance improvements are typically still far from the optimal performance achievable within given constraints. Being able to systematically screen for alternative designs and determine the optimal performance of a quantum system based on the optimal configuration of input variables, within a set of constraints—as will be described in this invention, is a precondition for the competitive and scalable deployment of resulting technologies.

In the management literature, an optimal implementation of a technology for a particular use case is referred to as the dominant design. Dominant designs can emerge in an evolutionary manner through trial and error or, alternatively, through systematic optimization, typically based on simulations of the behavior of the considered device system and its variants. Through the latter approach, the arrival at a dominant design—and subsequent large-scale dissemination of the technology in question—can be greatly accelerated. What is necessary to that end is a method for systematic optimization of the design—and a slightly different one for each use case—which in turn depends on having identified causal relationships between input variables and output variables (i.e. the system outcomes to be optimized).

To illustrate the importance of systematic optimization in the design of devices suitable for large-scale deployment, a comparison can be made to transistor development. Here, the importance of selecting an optimal or close-to-optimal material composition when seeking high performance is particularly pertinent. Early transistor prototypes such as the 1948 Bell Labs transistor prototypes functioned well enough to serve as a proof of principle demonstration of the amplification of electrical signals in solid-state environments. However, the performance of such prototypes—in terms of such factors as reliability, cost, and signal fidelity—was still far inferior compared to transistor designs developed several years later. These later, more advanced designs were based on more suitable materials compositions that were identified to yield superior performance. Concretely, the preferred materials composition evolved from Ge crystals with Sb doping at a concentration of around 1015 cm⁻³, as predominantly used in the 1940s, to Si crystals with B doping used by the late 1950s. If systematic modeling tools that captured the essential causal relationships governing transistor function had already existed during the late 1940s, then the effect of different input parameters (such as materials composition) on output variables of interest could have been determined more systematically. As a result, dominant designs for transistors for various use cases would have arrived earlier and dissemination would have occurred faster. This example illustrates how the ability to systematically predict, compare, and optimize the design implementations of new technologies are critical for large-scale dissemination and for the effective use and deployment of emerging technologies.

This insight can be applied to the emerging technology of coherently enhanced state transitions in quantum systems. As there are many different use cases for this emerging technology, as elaborated on below under the headings ‘modalities’ and ‘use cases’, each use case exhibits a different set of constraints and a different set of desired outcome parameters, requiring a dedicated optimization process for each use case to arrive at an optimum design respectively (including the identification of optimal configurations of input parameters in each case). The modeling and optimization method presented here addresses that need and presents a suitable approach to this problem.

### SUMMARY OF THE INVENTION

The invention relates to the design of energy transfer and conversion in quantum systems, and more specifically, to the use of quantum simulations and related modeling techniques to predict the quantum dynamical behavior of alternative system designs, to compare their performance, and to select the most desirable of such alternative designs.

Alternative variants of device systems, thus compared, differ based on direct, controllable input variables such as materials composition, environmental conditions, and stimulation characteristics, as well as indirect variables that follow from the direct variables such as the structure, mechanical, and optical behaviors of the materials that result from design choices. Subsequent comparison and evaluation of alternative variants of systems take place based on the ability to achieve desired outcomes according to pre-defined criteria. Such criteria can include, but are not limited to, a minimum rate of charge production, nontoxicity of materials, longevity of the device system, and materials within a certain price range.

In general, in one aspect, the invention features a computer implemented method for optimizing energy transfer and conversion in coupled quantum systems, and for creating corresponding device designs. The method includes the following steps. First, providing a database comprising input variables of one or more quantum systems in a lattice sample of a single material or an alloy or a composite material. Next, modeling an initial crystal structure of the lattice sample at a first set of environmental parameters via a computing engine. Next, adding a dopant to the lattice sample and determining a new equilibrium state of the lattice sample at a second set of environmental parameters via the computing engine. This step is repeated if more than one dopant is added. Next, determining lattice-related oscillator characteristics such as phonon-modes and photon absorption in the new equilibrium state of the lattice sample via the computing engine. Next, estimating state transition rates for a first quantum system in the lattice sample in the absence of any strong coupling to a second quantum system via the computing engine. Next, determining presence of any coupling and coupling strength of the first quantum system to the second quantum system via the computing engine. Next, providing coherent stimulation of the lattice sample via the computing engine, thereby populating oscillator modes that the first quantum system and the second quantum system both participate in. Next, determining presence of coupling and enhancement of coupling strength of the first quantum system to the second quantum system after the coherent stimulation of the lattice sample via the computing engine. Next, determining energy transfer and conversion dynamics as a result of the coherent stimulation of the lattice sample and the enhanced coupling of the first quantum system to the second quantum system via the computing engine. Finally, determining output variables of the energy transfer and conversion dynamics between the first quantum system and the second quantum system in the lattice sample via the computing engine.

Implementations of this aspect of the invention may include one or more of the following features. The direct and indirect input variables include compositions and structure of the single material or alloy or composite material of the lattice sample, energy levels, state lifetimes, and multipolarity of the quantum systems in the lattice sample, geometric arrangements of the quantum systems in the lattice sample, lattice-related oscillator characteristics, and characteristics of the coherent stimulation of the quantum systems in the lattice sample. In one embodiment, the coherent stimulation is carried out by laser irradiation of the sample lattice and the characteristic of the coherent stimulation comprise a laser wavelength λₚ, pulse energy Eₚ, pulse length tₚ, repetition rate rₚ, and spot size Aₚ. Each set of environmental parameters comprises a temperature, a pressure, an equilibrium time, and optionally an applied electromagnetic field. The method may further include modelling formation and diffusion of dopant-stabilized vacancies in the new equilibrium state of the lattice sample at a third set of environmental parameters via the computing engine, after the addition of the dopant to the lattice sample. The output variables include form of energy (kinetic or potential), and amount of energy released from the lattice sample as result of the coherent stimulation applied to the lattice sample, overall energy balance ΔE (energy released from the sample divided by energy applied to the sample), and list of reaction products and particles. The method may further include substituting the dopant with a different dopant or adding a different dopant and reiterating the above-mentioned steps via the computing engine. The quantum systems comprise one or more of nuclei, atoms, ions, and molecules. The equilibrium state of the lattice sample and phase stabilities (including the lattice structure, and vibrational modes, among others) are calculated with a density functional theory (DFT)-based method. The DFT-based method includes all of its variants, and all of its equivalents known by different names, as well as all the semi-empirical methods where some parts of the total energy function are approximated and some parts are simulated, such as, Quantum Espresso software package, Atomic Simulation Environment (ASE) software packages, VASP with Phonopy software packages, or an open-source stochastic self-consistent harmonic approximation (SSCHA) software package. The method may further include calculating background electron density in the vicinity of the dopant via the DFT-based method. The method may further include estimating background electron density in the vicinity of the dopant based on experimental and theoretical values given in the literature. The energy transfer can be accompanied by forms of energy conversion such as upconversion and downconversion if there is a suitable configuration of donor systems and acceptor systems (also referred to as receiver systems) that enable such dynamics.

In general, in another aspect the invention features a non-transitory computer-readable storage medium containing a computer program for optimizing energy transfer and conversion in quantum systems, and for creating corresponding device designs. The computer program when executed by a computing processor includes:
(a) accessing a database comprising input variables of one or more quantum systems in a lattice sample of a single material or an alloy or a composite material;
(b) modeling an initial crystal structure of the lattice sample at a first set of environmental parameters;
(c) adding a dopant to the lattice sample and determining a new equilibrium state of the lattice sample at a second set of environmental parameters;
(d) determining lattice-related oscillator characteristics such as phonon-modes and photon absorption in the new equilibrium state of the lattice sample;
(e) estimating state transition rates for a first quantum system in the lattice sample in the absence of any strong coupling to a second quantum system;
(f) determining presence of any coupling and coupling strength of the first quantum system to the second quantum system;
(g) providing coherent stimulation of the lattice sample, thereby populating oscillator modes that the first quantum system and the second quantum system participate in;
(h) determining presence of coupling and enhancement of coupling strength of the first quantum system to the second quantum system after the coherent stimulation of the lattice sample;
(i) determining energy transfer and conversion dynamics as a result of the coherent stimulation of the lattice sample and the enhanced coupling of the first quantum system to the second quantum system; and
(j) determining output variables of the energy transfer and conversion dynamics between the first quantum system and the second quantum system in the lattice sample.

### BRIEF DESCRIPTION OF THE DRAWINGS

Referring to the figures, wherein like numerals represent like parts throughout the several views:

FIG. 1 illustrates schematically a process of de-exciting quantum system A while exciting quantum system B through a temporarily enhanced coupling between A and B (flipping two qubits A and B from states |1> and |0> to states |0> and |1>, respectively);

FIG. 2 illustrates schematically the process of accelerating the nuclear quantum state transition from the 14 keV |Fe-57*> excited state to the |Fe-57> ground state by coupling to another Fe-57 nucleus;

FIG. 3 illustrates schematically the process of accelerating the nuclear quantum state transition from the |D₂> state to the |He-4> state by coupling to another He-4 nucleus and inducing the reverse |He-4> to |D₂> transition;

FIG. 4A illustrates schematically the process of accelerating the nuclear quantum state transition from the |D₂> state to the |He-4> state by coupling to a U-238 nucleus in the sample and inducing the |U-238> to |U-238*> transition which results in a subsequent disintegration via 28 MeV charged particle emission;

FIG. 4B depicts data obtained from an experimental configuration with a D-implanted Ti and U sample, testing the quantum dynamics represented in FIG. 4A, which resulted in 28 MeV charged particle detection;

FIG. 5 depicts an overview diagram of the system for modelling energy transfer in coupled quantum systems, according to this invention;

FIG. 5A is a schematic diagram of the process for modelling energy transfer in coupled quantum systems, according to this invention;

FIG. 5B depicts an exemplary apparatus for generating and monitoring energetic particle emission via phonon-mediated nuclear excitation transfer;

FIG. 6A is a flow diagram of the process for modelling energy transfer in coupled quantum systems, according to this invention;

FIG. 6B is a detailed flow diagram of step A of the process of FIG. 6A;

FIG. 7 depicts an example of phonon mode prediction for a Si lattice in a plot of density of states versus frequency from Density Functional Theory (DFT)-based lattice simulation techniques (Phonopy and VASP) along with the input parameters used;

FIG. 8A depicts an example of nuclear excited states of isotopes Li-6 and He-4 as obtained from NuDat data via a Python wrapper library;

FIG. 8B depicts an example of nuclear states as predicted by NuShellX code and are to be considered in step F of the process of FIG. 6A;

FIG. 8C depicts the table of nuclides, which represents the full repertoire of building blocks that the designer can draw on in considering lattice compositions in step F of the process of FIG. 6A.

FIG. 9 depicts schematically a power production device to be designed and optimized according to this invention;

FIG. 10A, FIG. 10B, and FIG. 10C depict schematically the energy transfer process in the embodiment of FIG. 9;

FIG. 11 depicts schematically spontaneous emission in an uncoupled lossy oscillator simulated according to the method of this invention;

FIG. 12 depicts schematically energy transfer between two coupled lossy oscillators simulated according to the method of this invention;

FIG. 13 depicts schematically temporary increase of coupling strength between two coupled lossy oscillators simulated according to the method of this invention;

FIG. 14 depicts schematically upconversion resulting from increase of coupling strength between three coupled lossy oscillators simulated according to the method of this invention;

FIG. 15 depicts schematically downconversion resulting from increase of coupling strength between three coupled lossy oscillators simulated according to the method of this invention;

FIG. 16 depicts an experimental set-up for observing the simulated energy transfer processes according to this invention; and

FIG. 17 depicts an exemplary computing system for the implementation of the computer code 415 of the present invention.

### DETAILED DESCRIPTION OF THE INVENTION

The present invention relates to the design of devices that involve energy transfer and conversion in quantum systems, and more specifically, to the use of quantum simulations and related modeling techniques to predict the quantum dynamical behavior of alternative system designs, to compare their performance, and to select the most desirable of such alternative designs. Alternative variants of systems thus compared differ based on direct, controllable input variables such as materials composition, environmental conditions (also referred to as boundary conditions), and stimulation characteristics, as well as indirect variables that follow from the direct variables such as the structure, mechanical, and optical behaviors of the materials that result from design choices. Subsequent comparison and evaluation of alternative variants of systems take place based on the ability to achieve desired outcomes according to pre-defined criteria. Such criteria can include, but are not limited to, a minimum rate of charge production, nontoxicity of materials, longevity of the device, and materials within a certain procurement price range.

### Definition of Terms

For the sake of consistent nomenclature, several terms used in this document are defined in this section. ‘Quantum systems’ are any physical systems at the nanometer and sub-nanometer scale that can absorb and release energy in a quantized manner (and thus can exhibit excited states and ground states, referred to as ‘states’). This includes, but is not limited to, atoms, molecules, and nuclei. Multiple quantum systems can be coupled through physical interactions that couple (some of) their states and can thus form larger, coupled quantum systems comprised of individual quantum systems. Such larger, coupled quantum systems are here referred to as ‘coupled quantum systems’ or ‘compound quantum systems’. Note that many coupled quantum systems are also quantum systems. Moreover, individual quantum systems and coupled quantum systems can be part of a larger lattice which is here referred to as a ‘sample lattice’ or ‘sample’. Note that the lattice does not necessarily have to be regular but can also be amorphous. Sample lattices can be part of larger systems that can be seen as devices whereas such devices can comprise multiple components that interact in a specific manner to achieve a specific purpose, per the device design. Such overall systems are here referred to as ‘device systems’, or simply ‘devices’. Couplings between quantum systems can be direct or indirect. In the case of direct couplings, physical dynamics in one quantum system directly impact physical dynamics in another quantum system. In the case of indirect couplings, the physical dynamics in one quantum system interact with an oscillatory mode that also interacts with the physical dynamics in another quantum system. In the text below, ‘oscillators’ and ‘oscillator modes’ describe any oscillators and their modes that can cause an indirect coupling between the states of at least two quantum systems in the device. Such oscillators can take the form of coherent photons, phonons, or plasmons, among others, each of which exhibits physical mechanisms of interacting with quantum systems such as atoms and nuclei through basic principles e.g. electromagnetic and mechanical/relativistic principles, as has been studied in detail for each combination of major types of oscillators and major types of quantum systems. The corresponding literature that studies such basic interactions also describes the determination and estimation of coupling strengths between quantum systems under given circumstances i.e. in the presence of different oscillators that are shared across quantum systems.

In this invention, emphasis is placed on the coupling strengths that result from shared oscillator modes that interact with multiple quantum systems, their enhancement by external stimulation, and further enhancement by collective quantum effects such as superradiance. The exact nature of the interactions that underlie these couplings (as laid out above) and therefore the types of oscillators used to cause enhancement of couplings (e.g. coherent phonons, photons, plasmons) is secondary as long as the resulting coupling strengths are strong enough to cause significant effects. In other words, the method and system described here is agnostic towards the detailed nature of the couplings between quantum systems. The key issue of interest is typically whether the couplings between the states of different quantum systems—and especially after all possible enhancements have been taken into account—are strong enough to result in modified dynamics (compared to weak or unenhanced couplings) with observable outcomes. The optimization and design principles described here apply across devices whose operations are based on different types of couplings and resulting energy transfer between quantum systems.

### Excitation and De-Excitation: State Transitions of Quantum Systems

Quantum systems, such as atoms, molecules, nuclei, can be in an energetic ground state or in an energetic excited state. Excited states are characterized by the amount of energy held by the quantum system in the respective state (compared to the ground state), by the lifetime of the state, and in some cases by other aspects such as the multipolarity of the state. Depending on the quantum system and state, lifetimes can range from less than nanoseconds to more than millions of years. Because of the probabilistic nature of quantum systems, lifetimes of states are not fixed values but exhibit some randomness according to probability distributions whose means are referred to as half-lives of the respective states. Such half-lives correspond to (by inverse proportionality) mean state transition rates which are here simply referred to as state transition rates or transition rates. If the state transition of a quantum system in question corresponds to what is more widely described as a reaction, then the state transition rate corresponds to the reaction rate (e.g. alpha particle emission from an excited nucleus can be equally seen as a state transition and as a nuclear reaction, depending on the viewpoint and preferred nomenclature). The probabilistic distribution of observed de-excitation times of a quantum system A (and thus the state transition rate or, in other words, the reaction rate where applicable) is impacted by the number and the strengths of couplings from the state of that quantum system A to states of other quantum systems which can—individually or collectively—accommodate the energy of said quantum system A. In the case of a large number of weak couplings, the so-called Fermi's Golden Rule applies, and de-excitation times follow an exponentially shaped probability distribution.

### Affecting Rates of State Transitions Through Couplings

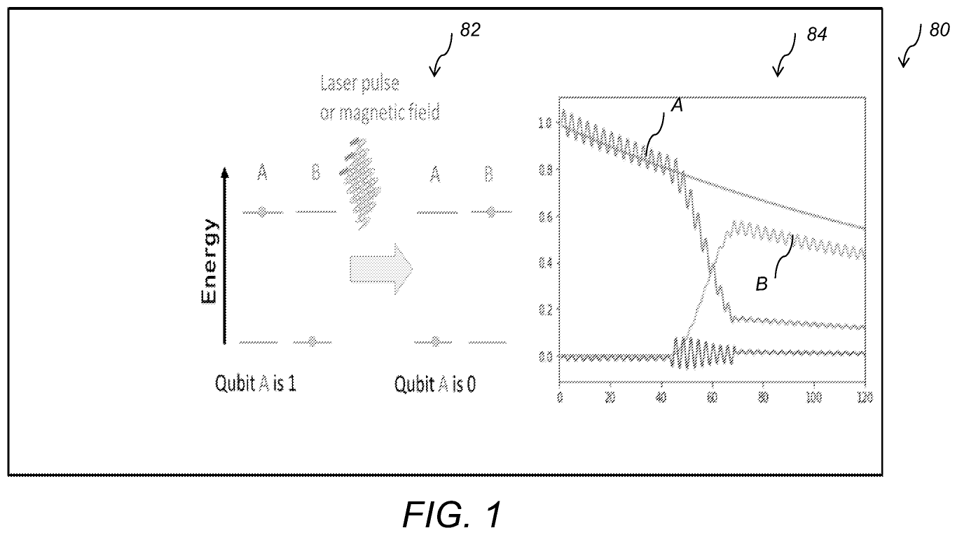

In the case of a single strong coupling to another quantum system that can accommodate energy from quantum system A, de-excitation follows a nonexponential probability distribution such as a sinusoidal distribution where state occupation probabilities oscillate back and forth between the two strongly coupled quantum systems (known as Rabi oscillations). It follows that de-excitation times can be affected by changing the strengths of couplings between quantum systems of interest. This effect is employed in quantum computing applications where “flipping a qubit”, i.e. switching a qubit from |0> to |1> or from |1> to |0>, is accomplished by temporarily increasing a coupling to 5 the qubit, thereby accelerating excitation or de-excitation (depending on the configuration). As shown in FIG. 1, qubits A and B are initially in state where A is |1> and B is |0> and are flipped to a state where A is |0> and B is |1> by temporarily increasing the coupling between qubits A and B via a laser pulse or an applied magnetic field. The increase in coupling strength leads to a transfer of excitation from quantum system A to quantum system B. A qubit is a quantum system per the above definition and can be physically implemented in a variety of ways, whereas the principles and methods described here are independent of the specific physical implementation.

### Energy Conversion Alongside State Transitions

Whereas in quantum computing applications the focus lies on the information content of quantum systems (qubits), in energy applications the focus lies on the amount of energy (as measured in eV) held by quantum systems. Energy associated with quantum systems can take different forms, specifically: potential energy and kinetic energy. In some systems, the de-excitation of a quantum system from an excited state to a lower excited state, or to the ground state, corresponds to a conversion of energy from potential energy to kinetic energy.

Potential energy in one modality represents the energy attributed to electrons whose constellation forms the excited state of an atom or molecule. In another modality, potential energy represents the binding energy attributed to the nucleons whose constellation forms the excited state of a nucleus. In turn, kinetic energy describes the energy of a moving quantum system such as an emitted photon or other particles to which released potential energy gets transferred.

### Acceleration of State Transitions as Acceleration of Reactions

Different energy levels of a quantum system, i.e. excited states and the ground state, can be thought of as different energetic configurations of components of that system, e.g. the configuration of electrons in atomic level quantum systems and the configuration of nucleons in nuclear level quantum systems.

A state transition of a quantum system, e.g. from a higher energetic state to a lower energetic state, can then be thought of as a rearrangement of electrons, or nucleons respectively, from one configuration to another configuration. From this perspective, certain nanoscale dynamics that are sometimes referred to as ‘reactions’ de facto fall under the more general umbrella term ‘state transitions’. Consequently, the above considerations apply to them. In this vain, Physics Nobel laureate Julian Schwinger 10 pointed out that the nuclear reaction D₂→He-4 ought to be modelled as a nuclear state transition from a highly clustered four-nucleon-configuration (|D₂>—a quantum system in a metastable state) to a more compact and more stable four-nucleon-configuration (|He-4>—the same quantum system in a different, lower-energy state). The reaction rate associated with that nuclear reaction in a given environment is then conceptually equivalent to the state transition rate from one state of the quantum system to the other state. Consequently, an acceleration of the state transition rate of the quantum system, e.g. as a consequence of sufficiently strong couplings to nearby quantum systems that can accommodate the transition energy, is equivalent to the notion of accelerating the reaction rate.

### Fastest State Transitions Dominate Observables

In a macroscopic sample containing coupled quantum systems, such as a solid-state lattice which includes multiple individual quantum systems such as atoms, molecules, or nuclei, couplings between them, and corresponding energy transfer pathways—the dominant energy transfer and conversion pathways will be those with the fastest time constants i.e. the fastest transfer and transition rates.

Consequently, changing the composition and configuration of quantum systems in a sample as well as changing the nature and strengths of couplings between them (e.g. by increasing energies and populations of oscillators such as photons or phonons that coherently interact with the quantum systems), parameters of interest such as energy transfer and conversion rates, and associated state transition rates, in that sample can be changed.

### Comparatively Weak Couplings Can Have Large Effects as Long as Donor and Receiver Systems Are Sufficiently Resonant

A critical point is that in coupled quantum systems the mediating oscillators that provide coupling, or enhance coupling, do not need to be able to carry the same amount of energy as the energy that is transmitted between quantum systems as a result of their mediation. This is a critical difference between nonradiative energy transfer, as described here, and radiative energy transfer. In the latter case, an oscillator that couples to an excited state needs to be able to carry the entire quantum of the state transition energy of the quantum system. In other words, in the case of nonradiative energy transfer, as described here, the coupling, as expressed in eV, can be much weaker than the affected state transitions, as expressed in eV. This is particularly relevant when the quantum systems to be worked with are nuclei, as nuclear transitions tend to be in the keV and MeV range—whereas oscillator energies tend to be in the eV and meV range (and lower). The key here for applications is this: If separate quantum systems that are coupled are resonant, or close-to-resonant, (i.e. some of their states are resonant or close-to-resonant) and if the coupled quantum system can be maintained long enough, then the mediating oscillator (such as a coherent phonon or photon mode) does not need to be able to carry the entire state transition energy. If those conditions hold, then excitation energy can transfer nonradiatively between the quantum systems that are coupled even via comparatively low-energy mediators. Resonance between two quantum systems is achieved if the excitation energy (or a discrete part of it in the form of a state transition energy) held in one quantum system can be readily accommodated by another quantum system.

This approach—i.e. the transfer of large quanta of excitation via couplings mediated by comparatively low-energy oscillators—opens up a whole new world of energy-oriented quantum engineering.

When working with nuclei as quantum systems, this new domain can be referred to as nuclear quantum engineering, or as quantum-coherent nuclear engineering. The implication is more degrees of freedom in the exploitation of nuclear reactions and the design of corresponding systems. Effectively, this means that well-chosen arrangements of nuclei (and the potential nuclear reactions that they can undergo) can be instigated and triggered by the provision of the right kind of couplings, or coupling enhancements, between the participating quantum systems (e.g. via coherent phonons) i.e. couplings and coupling enhancements that lead to desired outcomes. This invention describes methods and systems for designing and optimizing such systems based on modeling and simulating their behavior, and specifically the dominant energy transfer pathways and corresponding reaction rates and reaction products in different variants of such systems, through integrated modeling of nuclear level and atomic level features.

### EXAMPLE I

### Accelerating |Fe-57*>→|Fe-57> Transition Via Coupled |Fe-57>→Fe-57*> Transition

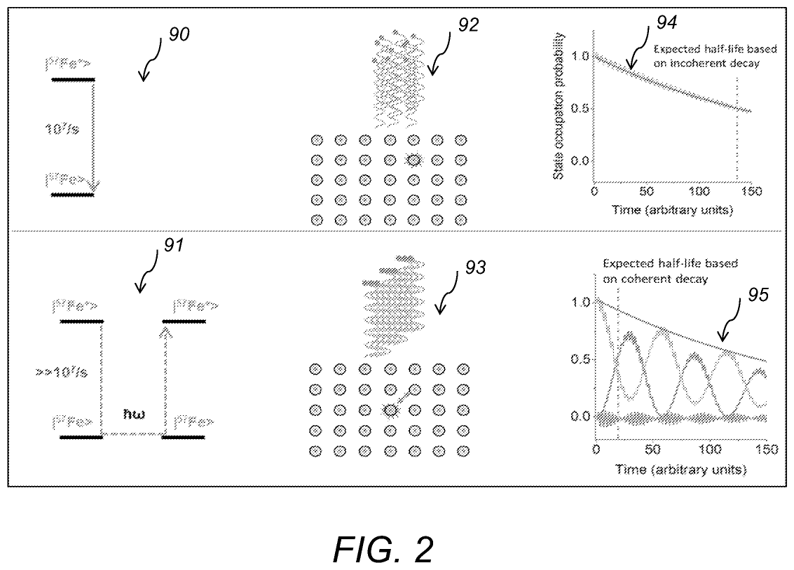

To provide an example for coherent oscillator-mediated nonradiative energy transfer of nuclear excitation consider a sample that comprises a lattice of Fe-57 nuclei 90 at ambient environmental conditions where a Fe-57 nucleus A is deliberately excited into its 14.4 keV excited state |Fe-57*> by an X-ray laser, as shown in FIG. 2. An individual Fe-57 90 nucleus in isolation (i.e. with no strong couplings to any specific receiver systems) would then de-excite with a mean time of 98 ns, as recorded in Eckhause et al. 1966. As shown in FIG. 2, adding incoherent photons 92 does not change this picture. However, adding coherent photons 93 where both the excited nucleus A and other Fe-57 nuclei participate in the same oscillator mode changes the situation qualitatively. As one or multiple couplings are provided to another nearby Fe-57 nucleus B,—e.g. by both nuclei participating in the same coherent oscillator mode—the mean de-excitation time of nucleus A decreases accordingly. Participating in the same coherent oscillator modes results in a much stronger coupling strength between nucleus

A and nearby nuclei, thus facilitating the transfer of energy from nucleus A to another Fe-57 nucleus (here: nucleus B as an acceptor nucleus). As a result of these dynamics, the de-excitation of nucleus A has become accelerated.

This example demonstrates how choosing the composition of nuclei in a lattice, their arrangement, and the type of stimulation that these nuclei undergo that leads to couplings between them is a way to affect their de-excitation and excitation characteristics or, in other words, to affect the energy transfer and conversion pathways in that sample.

### EXAMPLE II

### Accelerating |D₂>→|He-4> Transition Via Coupled |U-238>→|U-238 *> Transition

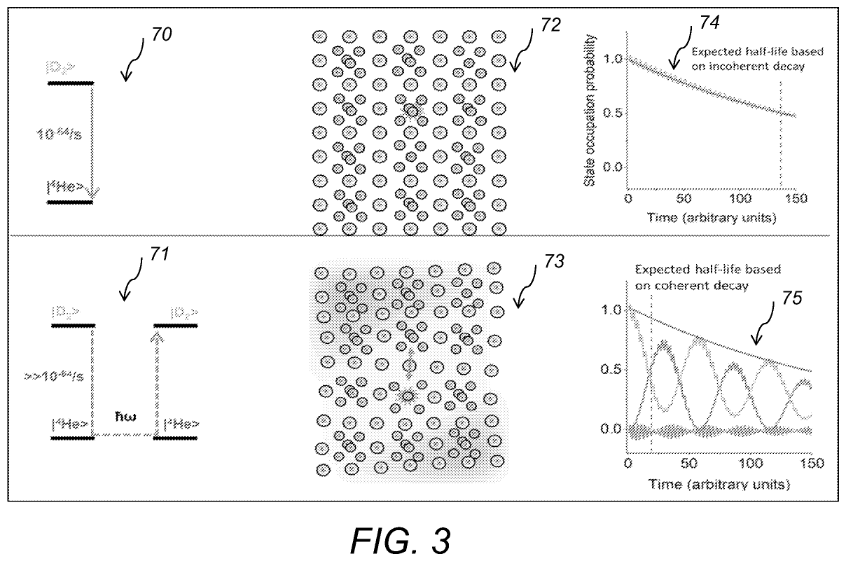

Another example pertains to a state transition of a quantum system that is traditionally viewed as a nuclear reaction. Referring to FIG. 3, consider a pair of deuterium nuclei 70 (deuterons) at proximity in the range of 70-100 pm (deuterium molecule or di-deuterium) in a metal hydrogen lattice with a high vacancy content (25% in the illustrated case). Per Schwinger such a deuteron pair is viewed as a single nuclear quantum system. Without stimulation of the sample via coherent oscillators such as coherent photons or coherent phonons, existing couplings between the D2 quantum system and other nuclear quantum systems in such a sample are extremely weak. In the absence of enhanced couplings, the individual quantum systems in the sample can be viewed as de facto isolated. Per Koonin & Nauenberg 1989, such a four-nucleon-configuration D₂ in isolation de-excites spontaneously to a more stable four-nucleon-configuration He-4 at an average rate of 10⁻⁶⁴/s, i.e. too slow to be observed in practice. This conventional (incoherent) state transition—which corresponds to the concept of spontaneous emission in quantum optics—results in the emission of either (1) an energetic proton plus an energetic tritium nucleus, (2) an energetic neutron plus an energetic He-3 nucleus, or (3) an energetic photon. In each case, potential energy is converted to kinetic energy as part of the state transition. To summarize, an isolated D2 pair transitions spontaneously to He-4 at an average rate of 10⁻⁶⁴/s, which is accompanied by a potential energy to kinetic energy conversion.

In the presence of shared coherent oscillator modes 73 which multiple nuclei participate in (a coherent phonon mode is here illustrated through lattice distortions), couplings between nuclei are enhanced. Stronger couplings enable the transfer of excitation to coupled resonant or close-to-resonant quantum systems. Here, the transfer of excitation 71 from D₂ to a nearby He-4 nucleus is shown in FIG. 3. The excitation of a compact four-nucleon-configuration |He-4> to a clustered four-nucleon-configuration |D₂> is resonant with the de-excitation of a clustered four-nucleon-configuration |D₂> to a compact four-nucleon-configuration |He-4>. The |He-4> quantum system is thus resonant with the |D₂> quantum system and amenable to excitation transfer, as long as a coupling between the two quantum systems and their respective nuclear states is sufficiently strong. Note that for coherent excitation transfer to occur, the strengths of the couplings do not need to amount to the large quanta of energy that is transferred. Even substantially lower energy couplings can mediate the transfer of comparatively higher energy quanta, as discussed above. Note that the example here describes a closed coupled quantum system where the energy remains within the coupled system and where the occupation probability moves back and forth coherently between the donor and the receiver quantum systems. An open quantum system where coherence is eventually broken and the transferring energy escapes into the environment, in the form of energetic particles,—thus making it amenable to further processing—is described below.

Now consider the same deuteron pair embedded in a lattice (metal hydride lattice) where couplings to nearby U-238 nuclei (present in the lattice as impurities or added via doping or alloying or layering) are provided through external stimulation, e.g. by providing coherent photons (photon-nuclear coupling) or coherent phonons (phonon-nuclear coupling) of sufficient flux and energy that interact with said nuclei and thus enable a coupled quantum system (see Hagelstein & Chaudhary 2013 on how to choose flux and energy levels for sufficient resulting coupling strength).

Referring to FIG. A, the energy released from a |D₂>→|He-4> state transition is about 24 MeV, and is transferred in this case not to a coupled He-4 nucleus (which can accommodate the transferred 24 MeV without imminent disintegration and energetic particle emission) but transferred to a coupled U-238 nucleus which accommodates the 24 MeV in an excited state |U-238*> (excited states, particularly rotational ones, are dense in that energy range in high-Z nuclei such as U-238). In other words, it can be resonant or close-to-resonant to the |D₂>→|He-4> transition. As a result, the stronger the coupling between a D2 pair and a nearby U-238 nucleus, the faster the |D₂>→|He-4> de-excitation transition as well as the corresponding U-238>→|U-238*> excitation transition. Moreover, if the coupling between the quantum systems is caused by a shared oscillator (e.g. coherent photon or phonon) mode which other quantum systems also participate in (which is the case in most macroscopic samples), then the collective quantum effect of superradiance further increases the effective coupling strength between participant quantum systems as a function of the number of relevant quantum systems participating. Since U-238* excited states at 24 MeV are not stable, a secondary reaction follows in short order after the excitation. The U-238* nucleus disintegrates incoherently via particle emission such as alpha emission (fast He-4 nucleus with about 28 MeV of kinetic energy, as can be determined from the corresponding mass defect) and the initially coherently transferred energy from the donor quantum system to the acceptor quantum system is thus eventually carried away incoherently in the form of kinetic energy. The incoherent energetic particle emission can be precluded if sufficiently strong couplings exist that make further coherent excitation transfer to another receiver system faster than the otherwise incoherent disintegration. In that case, the fastest pathways affecting the following receiver system need to be considered to predict overall observables, and so on.

To summarize, in the above described system, a |D₂> quantum system transitions to its |He-4> state (de-excitation) while simultaneously exciting a U-238 nucleus coupled to it (from its ground state |U-238> to one of many dense excited states |U-238*> near 24 MeV). This de-excitation state transition, and the corresponding excitation state transition, occur at a rate larger than the 10′4/s of the uncoupled spontaneous |D₂>→|He-4> de-excitation state transition that is described in Kooning & Nauenberg 1989. The excited U-238 nucleus subsequently decays via emission of an energetic He-4 nucleus i.e. an alpha particle. Overall, the process involves a potential-to-potential-to-kinetic energy conversion. The coupling of the otherwise de facto isolated nucleus to a resonant system leads to a change of the transition rate of the original quantum system as well as to a change of final observable products of the overall process. Using different nomenclature: the above described dynamics led to a change of the reaction rate and a change of the reaction products.

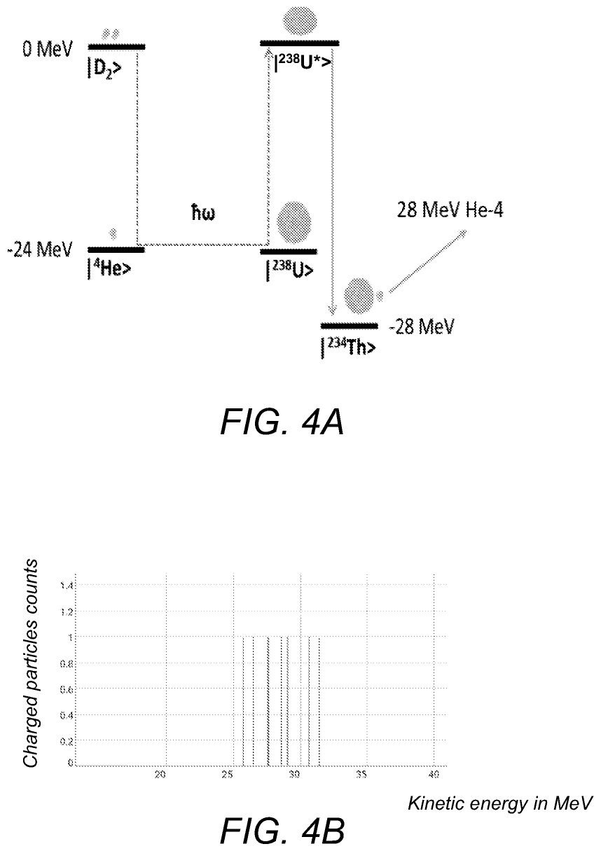

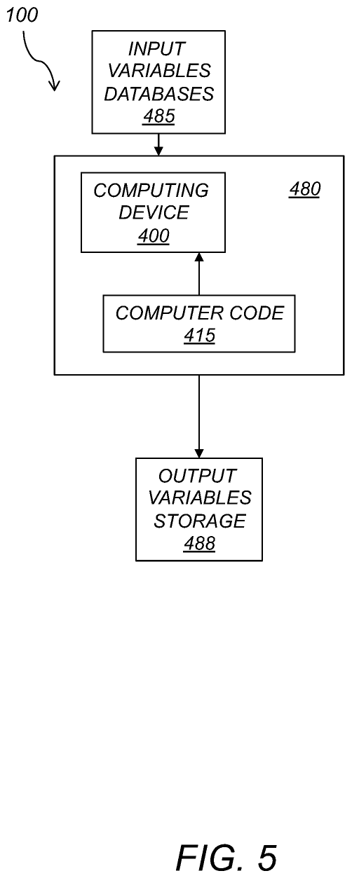

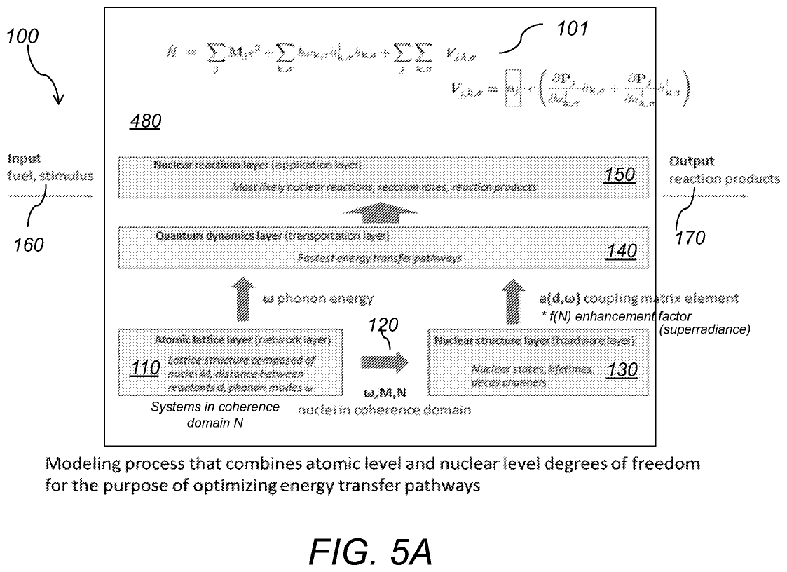

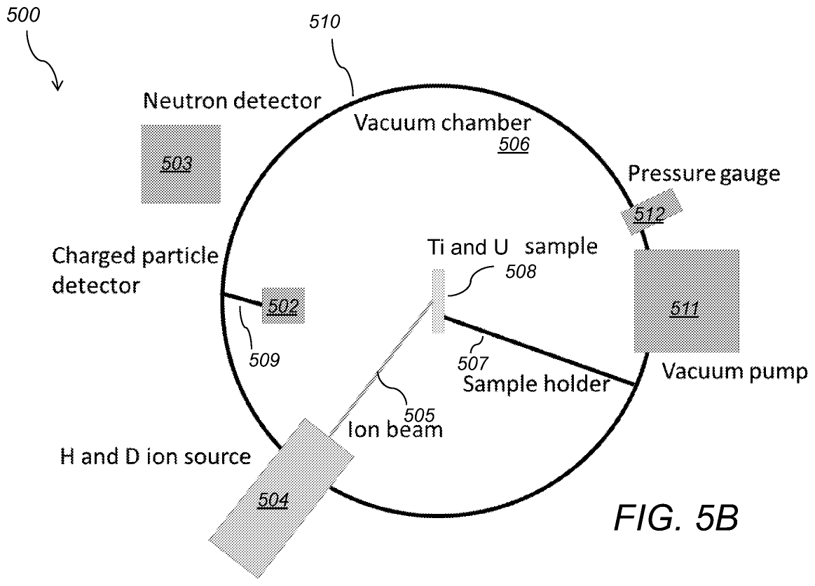

Experimental data exhibiting the observed 28 MeV charged particles in the above-described configuration is shown in FIG. 4B where the x-axis represents charged particle energy in MeV and the y-axis the number of particles detected by the charged particles detector at each energy bin. Data shown was obtained via the experimental apparatus depicted in FIG. 5B with a sample material of Ti with added U at a U:Ti ratio of at least 1:10^7. The corresponding energy level diagram is shown in FIG. 4A, depicting the nonradiative energy transfer of 24 MeV followed by the incoherent decay of the U-238 acceptor nucleus in its highly excited state via 28 MeV alpha particle emission (which then becomes detectable).

Specifics of this process such as rates and overall products—and thus the form and amount of energy released in a given window of time—depend on the nuclear states of the donor and acceptor systems and the coupling strengths between them, which in turn depend on their geometric arrangement, the type of stimulation that enhances coupling strengths, and the size of the respective coupled quantum system (i.e. the size of the coherence domain) as it impacts the extent of superradiant enhancement. It is these factors that the designer of an overall system can vary in order to arrive at an optimal implementation, given a set of desired outcomes. This invention in one aspect describes a method to conduct this optimization process systematically and to derive optimal device designs for different use cases from it.

The method described here in one aspect describes a computer implemented method for an accelerated path toward dominant design development for various use cases of energy transfer and conversion systems with coherently enhanced state transitions.

### The Energy Transfer System: Input Variables and Output Variables

This invention also describes a computer implemented optimization method, process, and implemented code for the design of systems in which quantum state transitions and thus energy transfer and energy conversion dynamics between coupled quantum systems are coherently enhanced.

Through a process of optimization, the invention allows designers of devices to determine the optimal choice of input variable values for reaching the parameter subspace of desired output variable values in the overall parameter space that results from relating input variables to output variables.

Such an approach—as described in this invention—is also known as a ‘rational engineering’ approach. More specifically, as described here, the approach can be referred to as ‘resonance engineering’, as it is concerned with the exploitation of resonances or near-resonances between quantum systems that are coupled, through the deliberate arrangement and stimulation of such quantum systems. The term is applicable both to resonances between atomic and between nuclear states of different quantum systems.

Referring to FIG. 5, and FIG. 5A, a system 100 for optimizing energy transfer and conversion in quantum systems by using quantum simulations includes databases 485 that contain input variables 160, a modeling engine 480, and storage 488 that contains the calculated output variables 170. The modeling engine 480 includes at least one computing device 400 or a computing architecture of networked devices and a computer code 415 that contains computer implemented instructions for optimizing energy transfer and conversion in quantum systems by using quantum simulations. As one exemplary embodiment, FIG. 5A exhibits the major physical magnitudes and relationships to be considered in the case of atomic nuclei as quantum systems coupled via shared coherent phonon modes. In this exemplary embodiment, the computer code 415 receives inputs 160 about the atomic lattice structures 110 of lattices of one or multiple species of atoms, and about the couplings 120 between atomic or nuclear states 130 of at least two such atoms based on methods of coupling the energetic states of nearby quantum systems such as atoms or nuclei. Atomic lattice structures 110 are lattice structures composed of nuclei M that have a distance d between the reactants and phonon modes at frequencies ω [as represented in the form of a vector/array]. Inputs 160 to such a code are compositions and structure of the single material or alloy or composite material of the lattice sample, the energy levels, state lifetimes, and the multipolarity of the quantum systems in the sample (e.g. of the nuclei); the dominant decay channels of relevant excited states of quantum systems in the sample (e.g. of the nuclei); the geometric arrangement of the quantum systems in the sample including the proximities between quantum systems in the sample as well as related effects such as electron screening corresponding to the free electron density in the lattice (whereas the geometric arrangement and free electron density are determined by the atoms' atomic properties and environmental conditions imposed); and the characteristics of stimulation such as the frequency and flux of coherent photons or phonons which in turn depend on the stimulation mechanism, e.g. the wavelength, energy, and pulse length of a stimulating laser. Outputs 170 to such a code are then the transition rates of the state transitions of interest in the sample as well as the final products that result from the dynamics of dominant transitions (after short-lived intermediate states have been traversed). Constraints on the overall optimization process imposed by the designer can include such aspects as the desired final products of the system (e.g. only charged particles in a particular energy range), the range of input materials (e.g. only materials within a certain procurement price range or excluding materials with high toxicity), and overall device characteristics (e.g. energy and power density of the device).

Referring to FIG. 5B, an exemplary apparatus 500 for generating and monitoring energetic particle emission via phonon-mediated nuclear excitation transfer, includes a sample assembly 510, a particle detector 502 and a H and D ion beam 505 generated by an ion source 504. Sample assembly 510 includes a vacuum chamber 506 and sample 508 supported on a sample holder 507 within the vacuum chamber 506. Generally speaking, in this exemplary apparatus 500, phonon-mediated nuclear excitation transfer leads to a change of nuclear reaction products (from a first nuclear reaction [energetic particles with a first energy] to reaction products of a second nuclear reaction/disintegration [energetic particles with a second energy]). The details of the exemplary apparatus 500 and other examples of exemplary apparatuses for generating and monitoring energetic particle emission via phonon-mediated nuclear excitation transfer are described in U.S. nonprovisional application Serial No. 15/733,950 filed Dec. 1, 2020 and entitled “System and method for phonon-mediated excitation and de-excitation of nuclear states”, the contents of which are expressly incorporated herein by reference.

It should also be noted that the embodiment illustrated in FIG. 5A is exemplary. Other embodiments of the system may utilize different sets and arrangements of atomic level and nuclear level data, variables, and calculations to the same end of optimizing energy transfer and conversion in quantum systems. The embodiment illustrated in FIG. 5A should therefore not be interpreted to be exclusive or limiting, but rather exemplary or illustrative.

### Integrated Atomic and Nuclear Codes With Optimization Function

The present invention describes the integration of multiple distinct techniques and codes to represent and simulate the behavior of coupled quantum systems, as described above, and allow for both manual and automated optimization of selected outcome parameters as a function of input parameters, and the corresponding design of high-performing devices. At the core of this invention in one aspect lies the combination and integration of atomic level modeling techniques and data with nuclear level modeling techniques and data, in order to optimize the performance of the above described device systems and, specifically, the dynamics of their constituent coupled quantum systems. Such coupled quantum systems extend over areas across at least several Angstroms and, in some cases, many nanometers. The description below focuses on an application of the method according to this invention that uses nuclei as the quantum systems whose dynamics are to be affected. Analogous principles apply when working with atomic level quantum systems.

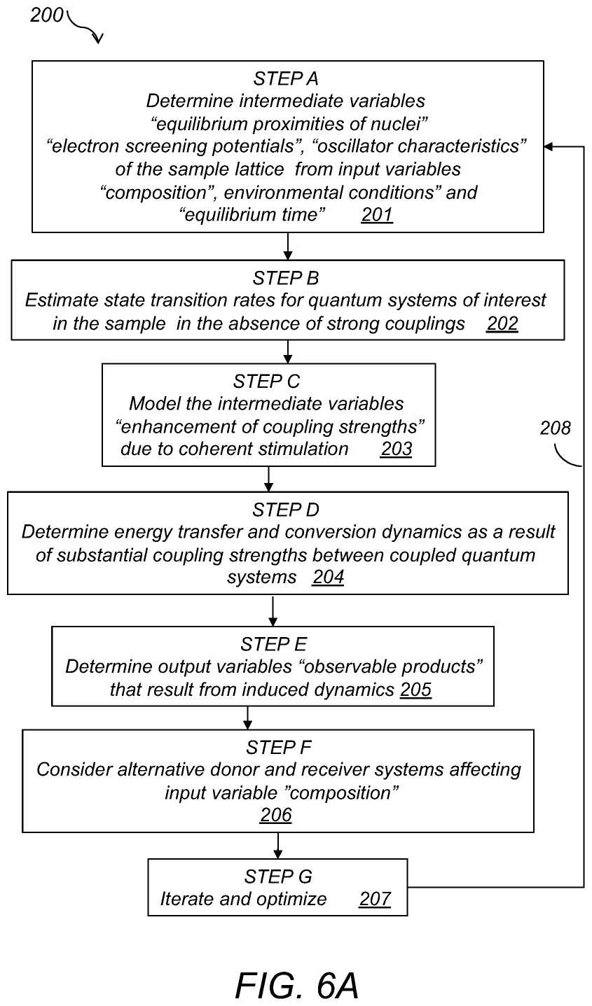

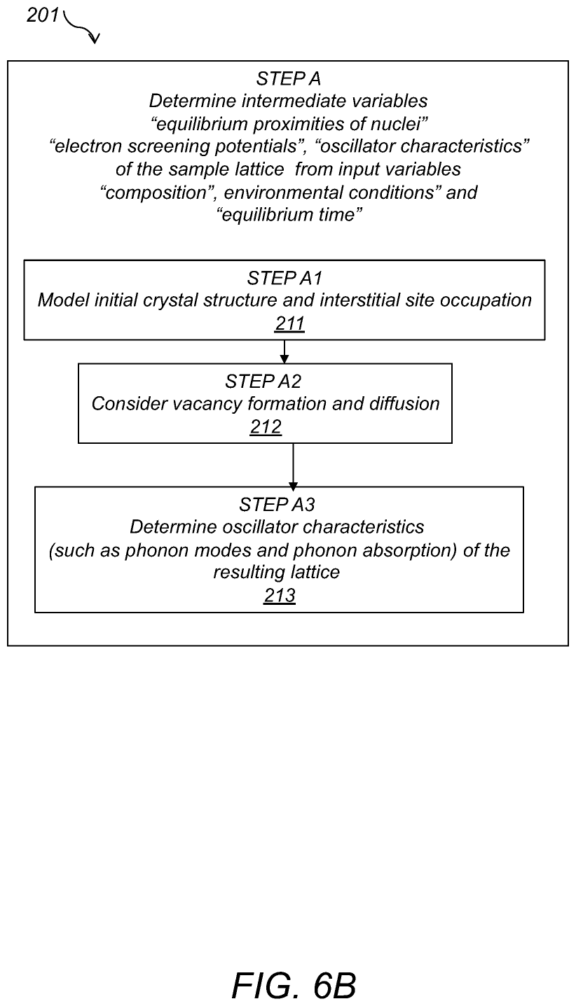

The overall modeling process of the computer code 415, as illustrated in FIG. 6A-FIG. 6B, is described in detail in sequential steps A-G below.

Step A: Determine intermediate variables “equilibrium proximities of nuclei”, “electron screening potentials”, “oscillator characteristics” (which, depending on the shared oscillators used, can be “phonon modes”, and “photon absorption”) of the sample lattice from input variables “composition”, “environmental conditions”, and “equilibration time” (201).

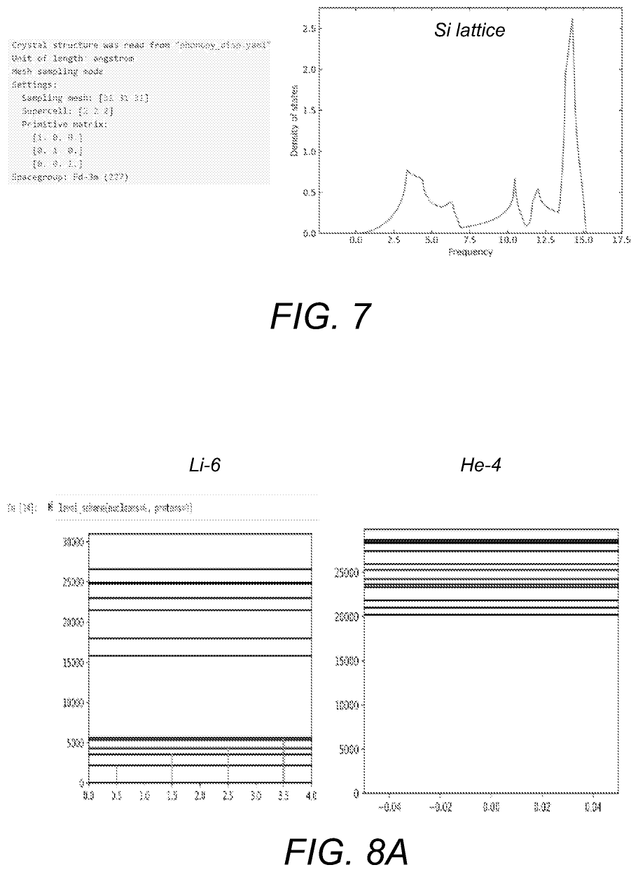

In one example, the intermediate variables are determined using a Density Functional Theory (DFT)-based method (which includes all of its variants, and all of its equivalents known by different names, as well as all the semi-empirical methods where some parts of the total energy function are approximated and some parts are simulated) such as Quantum Espresso software package, Atomic Simulation Environment (ASE) software package, or an open-source stochastic self-consistent harmonic approximation (SSCHA) software package. Shown in FIG. 7 is an example for the density of states of phonon modes with frequencies w from a simulated lattice based on DFT calculations.

Step A optionally includes the following sub-steps A1-A3.

Step A1: Model initial crystal structure and interstitial site occupation (211). This steps starts with a model of a pristine lattice of a single material (e.g. MₓA_{y}, where y=0) or alloy (e.g. MₓA_{y}, where y>0) at temperature T₁ and pressure p₁ as of equilibrium time t_{eq1}. Next, dopants such as hydrogen isotopes are added through pressure or electrolysis and the new equilibrium condition is determined at temperature T₂ and pressure p₂ as of equilibrium time t_{eq2}. Recommended starting materials include, but are not limited to, Pd, Ni, Li, Ti, Ta, W, Mg, Pb, Fe.

Note that ‘dopant’, as used here, describes the addition of a material to a lattice at low or high concentrations, including very high concentrations on the order of, or exceeding, the number of lattice nuclei in a given volume.

In one embodiment, the modeling process starts with the consideration of a metal lattice in a hydrogen gas environment at temperature Ti and pressure pi after equilibration time t_{eq1}, e.g. a single Pd crystal in the form of a Pd nanoparticle in a D gas environment at 2 bar and room temperature. In one embodiment, thermodynamic equilibria and phase stabilities are calculated with a DFT-based method such as Quantum Espresso and Atomic Simulation Environment (ASE) software packages or the open-source stochastic self-consistent harmonic approximation (SSCHA) approach. Background electron density in the vicinity of the deuteron pairs of interest are either also calculated as an extension to the above-described DFT-based modeling or estimated based on experimental and theoretical values given in the literature. One example of a background electron density calculation is described in Czerski et al. 2006.

Step A2: Consider vacancy formation and diffusion (212). In this step the formation and diffusion of hydrogen-stabilized vacancies are modeled at temperature T₃ and pressure p₃ as of equilibrium time t_{eq3}.

In one embodiment, diffusion modeling techniques are applied to model the diffusion behavior of D into the Pd lattice, as well as the formation of D filled vacancies on the surface of the Pd lattice (where thermodynamically preferred) and the diffusion of such vacancies into the bulk lattice. Examples of the vacancy stabilization and diffusion modeling techniques are described in Fukai 2003, Isaeva et al. 2011, Staker 2020, and Subashiev et al. 2020.

To achieve within a given volume the occurrence of a large number of deuteron pairs at close proximity (below 100 pm—see Nazarov et al. 2014), a large number of vacancies—on the order of 10% and more—is desirable so as to benefit from superradiance effects with a large number of participating quantum systems as |D₂>→|He-4> transitions are of interest in this case (participating in a shared oscillator mode). In the model of hydrogen-assisted vacancy formation and vacancy diffusion from the surface into the bulk, temperature and pressure can be varied so as to achieve the desired high vacancy content. To achieve sufficiently high vacancy content within a reasonable period of time t_{eq2}, pressure p₂ and temperature T₂ may have to be elevated from the starting values. An alternative way to accelerate the diffusion of D-filled vacancies is to increase the surface-to-volume ratio e.g. by making the size of nanoparticles smaller (e.g. below 10 nm). The model is used to determine a suitable combination of environmental parameters such as pressure p₂, temperature T₂, diffusion time t_{eq2}, and surface-to-volume-ratio (e.g. as represented in nanoparticle diameter d) that results in a PdₓVac_{y}D_{z} material with a Vac:Pd ratio of at least 1:10 and a D:Pd ratio of at least 8:10. In the vacancies, some deuteron pairs then exhibit a proximity in the range of 70-110 pm. PdD with such a larger number of vacancies is also known as Pd in a superabundant vacancy phase or delta phase. If a PdₓVac_{y}D_{z} material with a high vacancy content forms under the chosen condition and after time t_{eq2}, then the above-mentioned phonon mode calculations need to be updated to reflect what is now effectively a ternary material (treating the regular vacancies as an alloying component).

Step A3: Determine oscillator characteristics such as phonon modes and photon absorption of the resulting lattice (213).

Once the equilibrium lattice structure that results from the above-described treatments is known, further intermediate variables as described under “Step A” can be calculated such as phonon modes of the lattice and photon absorption, utilizing DFT-based codes as described above.

Step B: Estimate state transition rates for quantum systems of interest in the sample in the absence of strong couplings (202).

In one example, when the quantum systems are nuclei, the nuclear states available for each nuclide in the sample (intermediate variables) are identified and the state transition rates are modeled under the assumption of isolation (i.e. assuming the absence of strong couplings).

In one example, the expected state transition rate between the |D₂> state and the |He-4> state of the four-nucleon-configuration of deuteron pairs in Pd vacancies is calculated based on the proximity and the screening potential achieved in the lattice configuration, as determined in the previous modeling step. In one example, the expected transition rate is calculated, as described in U.S. provisional patent application No. 63/186,249. Since at this stage no couplings to interacting quantum systems are yet considered, this state transition rate corresponds to the transition rate of an isolated pair of deuterons at the given proximity and screening potential.

Step C: Model the intermediate variables “enhancement of coupling strengths” due to coherent stimulation (203).

In this step, the coupling strengths between all quantum systems of interest are determined under equilibrium conditions (intermediate variables). Next, the coupling strengths between all quantum systems of interest after stimulation via coherent oscillators are determined and stored in separate variables or a single array of values. In one example, the coherent oscillator is a laser with wavelength λₚ, pulse energy Eₚ, pulse length tₚ, repetition rate rₚ, and spot size Aₚ.

In one embodiment, stimulation is included in the modeling process that increases the coupling strengths between (initially weakly) coupled quantum systems. In one embodiment, the addition of a laser is considered that is directed at a normal angle at the sample lattice described above and where the quantum systems are nuclei The photons from the laser interact directly with the nuclei in the sample lattice (photon-nuclear interactions) as well as indirectly via plasmons and phonons (phonon-nuclear interactions) generated in the lattice as a result of the photon bombardment. The above-described interactions between nuclei and coherent oscillators lead to indirect (second and higher order) couplings between nuclei. Different laser configurations (such as those resulting in different pulse lengths) will cause different coupling strengths. The impact of the laser pulses on the sample can also be modified by applying nanostructured coatings to the sample that change/enhance the light-matter interaction. In the overall modeling process, the resulting photon-nuclear interactions and phonon-nuclear interactions—as a function of the laser parameters—are then calculated. Different nuclei participating in the same shared coherent oscillator modes results in enhanced coupling between such nuclei. Next, this enhancement of coupling strength caused by the coherent stimulation is calculated or estimated for each pairwise combination of nuclei in the sample. Estimating thus obtained enhancement of coupling strengths depends on the oscillator characteristics (e.g. coherent photon or phonon characteristics) as well as the nuclear state characteristics of the nuclei in the sample (e.g. state energy, state lifetime, state multipolarity). An example for estimating enhanced coupling strengths based on phonon-nuclear interactions is described by Hagelstein & Chaudhary 2013. Note that in addition to oscillator and nuclear state characteristics, coupling strength enhancement is amplified by superradiance effects which depend on the number of relevant participating quantum systems in the coherence domain of the coherent oscillators that enhance the coupling strengths of interest. Consequently, the extension of the coupled quantum system (i.e. its size) needs to be modeled to estimate the number of quantum systems N that participate in the coupled quantum system so as to determine additional enhancement from superradiance, which typically scales with N².

Based on the simulations of coupling strength enhancements, the laser parameters are chosen in such a way that an increase of phonon-mediated or photon-mediated couplings between nuclei of interest of at least 3 orders of magnitude results (e.g. from <10 neV to >10 μeV). The enhanced coupling strengths require that the previously estimated transition rates—obtained on the assumption of isolated (or only very weakling interacting) quantum systems—are adjusted for the quantum dynamics, including energy transfer and conversion dynamics, caused by the couplings.

Step D: Determine energy transfer and conversion dynamics as a result of substantial coupling strengths between coupled quantum systems (204).

In this step, the dominant energy transfer pathways—and resulting transition rates for quantum systems of interest—are determined from the previously determined coupling strengths (after all enhancements have been taken into account).

In one embodiment, adjusted state transition rates as well as energy transfer and conversion rates are calculated, now considering the enhanced couplings caused by the coherent stimulation of the sample. This process employs one or more quantum dynamical models. An example of such a quantum dynamical model 101 is shown in FIG. 5A. Simpler dynamical models that illustrate core principles of dynamics that can occur within coupled systems are discussed in more detail below. Given the enhanced coupling strengths, some of the resulting state transitions rates are now larger than the state transition rates considered for isolated quantum systems above.

In other words, in case of sufficiently strong couplings and available excitation transfer pathways, induced de-excitations of some quantum systems can coincide with excitations of other quantum systems—which in turn can trigger secondary reactions. For example, when the quantum systems are nuclei such triggered secondary reactions can include disintegration of the receiver nuclei. In that case, the range of secondary reactions can be determined by looking up from nuclear data, or by calculating from first principle based on the relevant nuclear physics literature, the expected behavior of thus excited nuclei upon receipt of the transferred quantum of energy.

Note that, depending on the lattice configuration and the parameters above, dominant pathways may involve upconversion or downconversion of energy redistributed in the coupled system (including energy released from nuclear reactions where applicable).

For details regarding upconversion and downconversion scenarios, consult the discussions below and corresponding simulations.

Next, all resulting state transition rates are listed and ranked. Practically relevant are only those state transitions that occur within a technologically meaningful timeframe (e.g. on the order of hours or less, as opposed to centuries and more). Moreover, those state transition rates that are much larger than others dominate the overall behavior of the sample.

As a sub step, feedback mechanisms need to be taken into account—if they occur at a significant level—to arrive at a final ranking of state transition rates. The ranking should consider all significant state transition rates in the device at equilibrium operation i.e. when the coupling strengths that drive the dominant state transitions are at their eventual equilibrium value. Particular focus is on processes where energy released by accelerated state transitions contributes to the further enhancement of relevant couplings which then further accelerate state transitions. This is for instance the case when energy quanta released from the de-excitation of atomic or nuclear transitions get downconverted to the level of phonons and then pump phonon modes in a way that further enhances phonon-mediated couplings and thus further enhances energy transfer between the coupled quantum systems, thereby further modifying state transition rates etc. If such feedback loops occur, they can be modeled and resulting equilibrium coupling strengths and rates determined—unless the feedback loop leads to the disintegration of the lattice, in which case no functional equilibrium is reached. In the latter case, the device in that particular configuration would be considered unsuitable for most applications and alternative configurations (i.e. alternative sets of input variables) preferable.

Step E: Determine output variables “observable products” that result from induced dynamics (205).

In this step, as output variables, secondary reactions are considered and the overall energy balance ΔE is determined (all energy released from the system divided by all energy applied to the system) and all reaction products are listed. As discussed, accelerated de-excitation through enhanced couplings often coincides with the simultaneous excitation of quantum systems that are coupled to (as a result of energy transfer and, in some cases conversion, in contrast to de-excitation and excitation only). This means that, once coupling strengths are enhanced via coherent stimulation of the system, some quantum systems in the sample that were previously not excited can get excited. In order to determine the behavior of the overall sample, this requires the modeler to consider how such newly excited quantum systems behave as a result of their excitation. Some such quantum systems remain in their excited state long enough for a subsequent excitation transfer to take place, effectively kicking the can down the road and requiring the consideration of the behavior of the subsequently excited quantum system, and so on. Some newly excited quantum systems will become unstable before subsequent energy transfer can take place. Such unstable excited quantum systems de-excite in a number of known ways, including by photon, neutron, or charged particle emission. In any case, the modeler needs to consider the expected behavior of excited states in quantum systems in the sample that were induced by coherent dynamics caused by the stimulation of the sample. For example, when the quantum systems are nuclei the behavior of excited states, including their state lifetimes, and expected decay pathways can be looked up in nuclear databases such as NuDat, from experimental and theoretical nuclear physics literature, or simulated via nuclear codes such as nuclear shell model codes (e.g. NuShellX, ANTOINE, among others).

To summarize thus far, this invention describes a modeling approach for a condensed matter sample that is stimulated to populate coherent oscillator modes in the sample that are shared across multiple quantum systems, which then cause quantum dynamics through enhanced couplings between quantum systems in the sample. The overall model of this process consists of an integration of several existing techniques, models, and codes in a well-defined and targeted manner. The overall input parameters to the system to be modeled are: (1) the materials composition of the sample (which impacts the resulting lattice or amorphic structures); (2) the environmental conditions under which the lattice forms or equilibrates (particularly temperature, pressure, and optionally applied electromagnetic fields); and (3) the type and characteristics of the coherent stimulation of the lattice to populate oscillator modes and enhance coupling strengths (e.g. wavelength, repetition rate, pulse length, and pulse energy of a laser). Intermediate output variables are the rates of state transitions (rates of reactions where applicable—see discussion of nomenclature above) as a result of the stimulation applied to the sample. Overall output variables are the form of energy (e.g. kinetic energy of alphas) and the amount of energy (e.g. 10³ counts of 28 MeV alphas per second per mm³ of sample) released from the system as a result of the stimulation applied to the sample.

To arrive at the overall output variables certain intermediate variables such as the rates of all significant state transitions are critical, which can be expressed as a vector/an array. The resulting state transition rates can then be ranked to identify the dominant state transitions (and corresponding reactions where applicable). This allows the designer to evaluate how the candidate system behaves from a macroscopic point of view and allows the determination of output variables such as the amount of energy converted by the device from one form into another.

Step F: Consider alternative donor and receiver systems, affecting input variable “composition” (206).

In this step, the effect of adding other species of quantum systems to the sample is considered.

For example, when the quantum systems are nuclei, other species of quantum systems added may be nuclei of a different species (i.e. different nuclides) added as dopants , or as alloy components or as composite material components (across a range of different concentrations z across simulation cycles).

In the above described embodiment, so far only a composition of two materials has been considered: Pd and D. In this case, couplings to be considered in terms of the enhancement they undergo pertain in particular to the nuclear states of deuteron pairs and the nuclear states of nearby Pd nuclei. However, if the sample also contains nuclei from other nuclear species, then further couplings as well as corresponding energy transfer and conversion pathways have to be considered. Other nuclei in the sample can be different with respect to their atomic species (i.e. element) and with respect to their nuclear species (i.e. isotope). Note that if other nuclei are considered in the sample that are a different element from those originally considered, and if the portion of those nuclei compared to the lattice nuclei is large enough, then the modeling of basic lattice parameters such as crystal structure and phonon modes—as described above—needs to be redone (per the corresponding steps listed above) before proceeding with downstream modeling aspects (note, for instance, that available phonon modes in a lattice can be impacted by doping). This is not the case if other nuclei that are added to the composition of the lattice only differ as isotopes. It is also not the case, if the portion of such other nuclei, even as different elements, is sufficiently small, e.g. <0.1%. In the latter case, the other nuclei can be considered as impurities or traces in the host lattice/the original sample. The earlier discussed modeling of lattice characteristics can then be expected to hold for all intents and purposes, despite the traces. However, what needs to be considered for certain in an adjusted sample with trace additives is what the dominant coupling strengths are, what the dominant energy transfer and conversion pathways are, and what the resulting dynamics are. Even if they are of the same atomic species, the other nuclei that are added exhibit different sets of nuclear states, with different energy levels and state characteristics, and with their own sets of couplings to other nuclei in the sample (i.e. from and to their nuclear states) and the corresponding potential for resonant or non-resonant excitation transfer behavior. If other nuclei are introduced to the sample, e.g. as traces, that exhibit strong couplings to nuclear states of other nuclei in the sample as well as resonant or close-to-resonant energy levels, then such other nuclei can act as prominent donor or receiver systems for energy transfer and conversion dynamics upon coherent stimulation of the sample.

The above implies that the modeler, after going through the presented modeling steps for obtaining state transition rates in a stimulated binary system such as PdD, can then iterate over the modeling process to consider the consequences of including other nuclei in the system as impurities, traces, dopants, alloying components, or composite material components. This can then alter dominant state transitions and corresponding energy transfer and conversion pathways, and as a result, also alter the forms and amounts of energy that get released from closed coupled quantum systems in the sample.

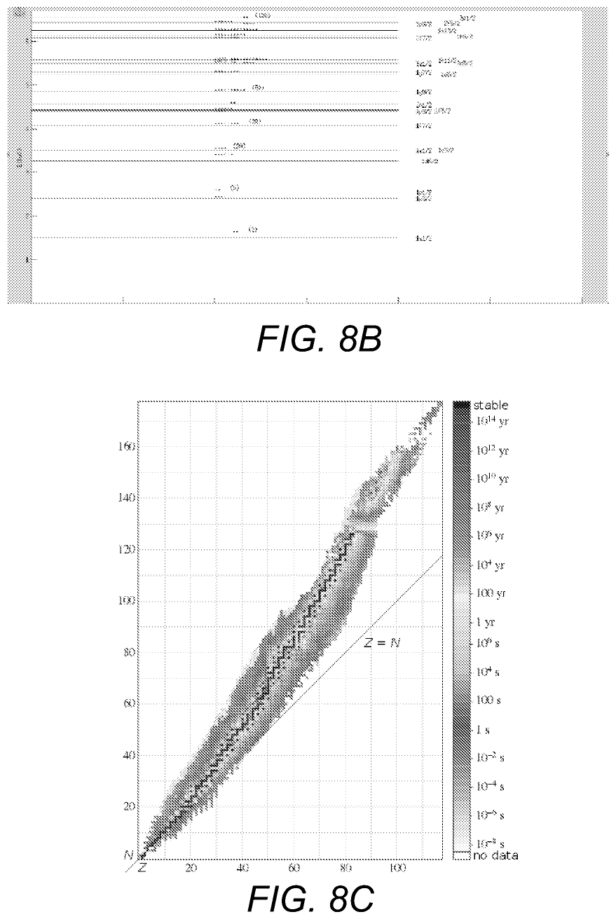

Shown in FIG. 8A are, as an example, nuclear excited states of isotopes Li-6 and He-4 as obtained from NuDat data via a Python wrapper library. In this figure, y-axes represent energy in keV and colors represent different decay channels. Shown in FIG. 8B are nuclear excited states as predicted by NuShellX code. Shown in FIG. 8C is the entire chart of nuclide, which represents the full repertoire of building blocks that the designer can draw on in considering lattice compositions. Again, each isotope selected to become part of the lattice in a given concentration can impact the lattice structure and lattice behavior (if the concentration is large enough) and exhibits different nuclear states and therefore different resonances and coupling strengths with oscillator modes.

In one example, the designer models the PdVacD lattice previously considered now with a 0.1% content of U-238 nuclei. In that case, the modeler considers not only couplings between deuterons and Pd host lattice nuclei, but also between deuterons and the U-238 nuclei that now form part of the lattice, as well as between U-238 nuclei and Pd host lattice nuclei. The modeler finds that the presence of U-238 nuclei as candidate energy transfer receiver nuclei impacts the de-excitation parameters of nearby deuteron pairs. Concretely, the state transition |D₂>→|He-4> (de-excitation) is accelerated if the released energy of 24 MeV is transferred to nearby U-238 nuclei as a result of enhanced coupling strengths, thereby causing the simultaneous state transition |U-238>→|U-238*> (excitation). The excited states of U-238 near 24 MeV are unstable and decay through particle emission, e.g. alpha emission. In that case, the model views the example discussed here as a sample with composition of Pd, D, and U-238 (as input variables, among others) which, under coherent stimulation and correspondingly enhanced couplings within coupled quantum systems in the sample, exhibits accelerated |D₂>→|He-4> state transitions and charged particle production from coherently excited and subsequently incoherently decaying U-238 nuclei (output variables, among others).

Step G: Iterate and optimize (207). In this step, the input variables are adjusted so as to optimize the output variables. In an exemplary embodiment, input variables to be adjusted include MₓA_{y}, T₁, p₁, t_{eq1}, T₂, p₂, t_{eq2}, T₃, p₃, t_{eq3}, λₚ, Eₚ, tₚ, rₚ, Aₚ, D and z, among others (as defined and introduced above). In one example, the optimized output variable is a maximized ΔE with, as a pre-defined criterion, the imposed constraint of not generating significant amounts of neutrons.

All the above steps can be implemented and tested separately with corresponding input variables, output variables, and intermediate variables. Eventually, the steps of the overall process can be chained together such that the model steps result in an overall model of the sample to be designed with global input variables and global output variables (and with further extensions of the device to be designed). Once the model is in place and integrated this way (and implemented in a computing engine), the model delivers de-excitation and excitation times for all state transitions of interest as a function of the chosen composition of the lattice, the boundary/environmental conditions, and the stimulation type and characteristics. Then, the input variables can be varied (manually or programmatically/automatically) and the effect on output variables observed.

As the modeler implements the above modeling and simulation process in an integrated manner, overall output parameters (such as the amount of energy leaving the closed system of the sample in a desirable form) can be expressed as a function of controllable input parameters (such as composition, environmental conditions, and stimulation characteristics). This then allows the modeler to predict the behavior of a large number of variations of the system to be modeled and iterate systematically across a range of parameters for the variables of the system that are subject to control. Such a comparison of system variations and optimization is carried out manually or automatically. In the automatic case, a large parameter space is traversed by the automatic execution of the model based on different input parameters and minima or maxima in the landscape of the output variables identified (depending on what output variable outcomes to be optimized for).

This allows the modeler to identify systems that, for instance, deliver optimal performance in maximizing released energy over required input energy, while meeting imposed constraints or requirements.

As the simulation and performance prediction process is automated as an iterative process to vary input variables systematically and automatically, key output variables of interest (or derived key performance indicators) across different input configurations, can be represented as a resulting “landscape” of outcomes. Minima and maxima of this landscape are points of interest that inform optimal outcomes i.e. an optimal set of input variable values for certain desired outcomes. Multiple output variables can be connected or related to one another e.g. if certain tradeoffs are preferred such as between overall energy gain and a desired energy range of emitted charged particles for instance.

For instance, the described system and process can be used to identify the most suitable isotopes, among a group of candidate isotopes, to be used in a system for efficient energy production resulting in heat. Additionally, different geometric arrangements of such isotopes to be used (as influenced by composition and boundary conditions during lattice formation) and their effects on the overall system dynamics and system outcomes, e.g. energy production rates, can be compared and evaluated. Geometric arrangements that may want to be considered, tested, and evaluated for their performance with the system and process described above include different nanostructures such as nanoparticles, nanosheets, thin-film configurations, nanopillars, nanoneedles, nanowires, nanotubes etc.

Further input and output variables can be added such as the materials cost of particular nuclear species chosen as input variables for the structure composition, the cost of obtaining and maintain certain environmental conditions (e.g. the cost of providing very high pressures), and the cost of the stimulation mechanism (e.g. the cost of ultrafast laser stimulation vs other mechanisms of coherent photon or phonon production).

Additional output variables can include the type of particles that get emitted from the reactions that result from the nuclear transitions that are predominant based on the predominant energy transfer pathways as well as their ultimate economic value (e.g. in terms of the released energy that can ultimately be harnessed and utilized this way). In one example, a device designer may find that substituting one alloying component in a sample for another leads to a 10% reduction of the overall energy balance (i.e. released output energy/applied input energy) but to a 90% cost reduction of the sample. Depending on the specifics of the pre-defined criteria, the device designer may then find that the sample with the substituted alloying component is preferred for her device design. Moreover, constraints can be imposed on input variables e.g. to include as candidate nuclear species only materials that are procurable within a certain price range or that do not exhibit certain hazards. Similarly, output variables can be constrained e.g. by only accepting as valid outcomes predominant nuclear transitions that lead to charged particle emission and not neutron emission. This way, the device system derived from the simulation and optimization process can be designed in such a way that from the perspective of the designer optimal outcomes are achieved (or near-optimal outcomes), i.e. optimal outcomes along the output variables considered to be most relevant, from selecting and adjusting a set of candidate input parameters to choose from across the input variables.

Constraints or requirements may also include, as examples, releasing at least 99% of the released energy as charged particles in the 1-5 MeV range, requiring nontoxic materials as part of the sample composition, and only requiring forms of stimulation that can be provided by commercially available means within a certain price range (e.g.

commercially available laser systems), among others. Similarly, searches and optimizations can be conducted for other use cases with their own respective requirements and constraints.

In scaled-up versions of device designs intended for dissemination and extensive deployment, the methods for producing coherent stimulation (such as coherent photons or phonons) considered during earlier design iterations may be substituted through comparable, but more cost-effective methods that yield similar results with respect to the resulting oscillator modes and the corresponding increase in coupling strengths. Besides laser stimulation, this includes—but is not limited to—thermal simulation, electrical stimulation, magnetic stimulation, or mechanical stimulation/pressure changes. In general, the process of modeling, simulation, performance prediction, and optimization described here is agnostic toward the specific stimulation method employed as long as, as a result of applying the chosen stimulation method, suitable oscillator modes get populated in the lattice which enhance couplings between quantum states of quantum systems of interest (resulting in a suitable vector/array of equilibrium coupling strengths to be used in the subsequent simulation of energy transfer and conversion dynamics).

The modeling process described above through steps A-G described the simulation and optimization method according to this invention with provided examples where the quantum systems whose quantum states got coupled as a result of enhanced couplings comprise atomic nuclei. The method applies analogously to coupled quantum systems where the quantum systems comprise atomic level quantum systems such as atoms or molecules.

Note that it is understood that in the above, any mention of a variable or value may also refer to a sequence or an array of variables or values, as is common in the arts.

### Modalities

In this section, different modalities of nonradiative excitation transfer and conversion are discussed. The discussion centers on exemplary embodiments where the quantum systems of interest are nuclei, whereas the generalized principles apply to all quantum systems as defined above.

Enlarging the degrees of freedom for device designers: At the most basic level, the invention presented here provides device designers with a way to greatly extend the degrees of freedom available to them in the design of useful devices. Even if only a single excitation transfer step is considered (i.e. transfer from a first type of quantum system to a second type of quantum system) this means that energy from one kind of de-excitation transition is redirected to another de-excitation transition (the one associated with the receiver quantum system). In the case that de-excitation takes the form of a nuclear reaction, for instance, this means that a nuclear reaction A (e.g. D+D→He-4) can lead to an outcome typically only associated with a nuclear reaction B (U-238*→Th-234+α). This is particularly attractive if nuclear reaction A exhibits certain attractive features such as beneficial chemical properties or comparatively easy procurability but not necessarily attractive reaction parameters and reaction outputs such as the typical reaction rates and reaction products for that reaction (e.g. neutrons). However, by redirecting the released energy through nuclear energy transfer editing i.e. the editing and optimizing of energy transfer pathways, as described here, overall reaction parameters and products can be changed, leading effectively to an initial reaction A exhibiting modified de-excitation parameters and products of a secondary reaction B. If that secondary reaction has more desirable products than the initial one (e.g. charged particles instead of neutrons), then the overall performance of a corresponding device is improved, compared with a device that does not make use of nonradiative energy transfer to that end.

Changing the accessibility of otherwise out of reach reactions: Of specific interest are those reactions that are typically not considered useful for applications and that now become useful and within reach by means of accelerating their rates. An exemplary case is the D+D fusion reaction, which is typically seen as less attractive than the D+T fusion reaction due to its lower cross section. Specifically, the estimated fusion rate at ambient temperatures and pressures is estimated as 10⁻⁶⁴/s per deuteron pair in a D₂ configuration (Koonin & Nauenberg 1989). However, as shown in US provisional patent application No. 63/186,249, an enhancement of more than 30 orders of magnitude can be expected as a result of the kind of coupled dynamics described here, as long as suitable acceptor quantum systems are built into the sample composition and can be sufficiently coupled to. Moreover, as also shown in U.S. provisional patent application No. 63/186,249, an enhancement of up to 20 orders of magnitude can be expected as a result of high electron density surrounding a deuteron pair such as in vacancies of metal hydrides such as PdD (with screening potential Uₑ of 150 eV and more). Combined, this leads to an overall enhancement of more than 50 orders of magnitude and thus to observable reaction outcomes in macroscopic samples (note that a cubic centimeter of PdD can contain 10^23 deuteron pairs and more). Laying out the accessibility of the D+D reaction via coupled nuclear states across atomic scales—and in some cases across macroscopic scales—emphasizes the importance of a simulation and optimization process that takes into account the full range of mechanisms presented here and presented in U.S. provisional patent application No. 63/186,249, both at the atomic and at the nuclear level, and stretching across them as is needed in the case of coupled quantum systems with interacting nuclear states across different nuclei. It also emphasizes the importance of the device designer considering the kinds of reactions that become feasible when embedded in condensed matter surroundings that can become coupled quantum systems with temporarily enhanced couplings. Specifically, for D+D fusion reactions to become feasible under ambient conditions, choosing inputs such as the following parameters are advised: high electron screening (preferably>150 eV) and close proximity of nuclei (preferably <100 pm) as well a high-energy phonon modes shared between D2 and other nuclear quantum systems (especially acceptor nuclei) in the lattice (preferably > 1 THz), large phonon coherence domains (preferably >5 nm coherence length), and resonant or close-to-resonant nuclear states between nuclear reactants (donor quantum systems) and acceptor nuclei (receiver quantum systems) within coupled quantum systems (i.e. coupled nuclei in that case). Suitable candidate compositions include PdVacD systems with high Vac content (between 10% and 25%) and high D:Pd ratio (>8:10) and added materials that can serve as stepping stones for downconversion such as Ag nuclei (see discussion below).

A secondary consideration that can enter into the design of samples that draw on energy released from the D+D reaction is directed at the possibility of out-of-equilibrium modes of operation such as adding reactants into the sample as well as removing reaction products from the sample during operation. Both processes are possible e.g. through diffusion. This can be achieved by changes in the environmental parameters such as increased gas pressure, increased temperature and is also affected by the sample composition and geometry, e.g. nanoparticles and thin films with large surface areas facilitate diffusion of reaction products into and out of the system.

Sequences of excitation transfer (daisy chaining): Energy transfer can also occur repeatedly (i.e. from a first type of quantum system to a second type of quantum system to a third type of quantum system etc.), thus allowing for a daisy chaining of acceptor quantum systems (and their possible induced reactions upon receipt of excitation) as long as transfers happen fast enough before the incoherent decay of one of the involved states occurs. This can be considered as the creation of “chains of excitation transfer” i.e. where excitation moves across a sequence of different species of receiver quantum systems before incoherent de-excitation takes over. What results are secondary reactions, tertiary reactions, and so on, induced by a primary reaction. Effectively this leads to a change of reaction rates and reaction products compared to what would have been considered the outcomes of the primary reaction in isolation. The possibility of sequencing excitation transfer—and corresponding reactions to be expected as a result—further extends the options available to device designers. Specifically, it allows for making use of what can be called ‘stepping stones’ i.e. quantum states that can act as intermediaries e.g. in a repeated downconversion process or upconversion process (see discussion below). For instance, a 24 MeV quantum from a |D₂>→∛He-4> state transition can downconvert to a commensurate number of exemplary 14 keV excited states of receiver systems (with some exchange of energy with the oscillator to make up remaining mismatch). The 14 keV excited states can then downconvert further to a commensurate number of states below 1 eV, resulting in what is largely an absence of ionizing radiation.

Upconversion: Note that energy transfer can be accompanied by forms of energy conversion such as upconversion and downconversion if there is a suitable configuration of donor systems and acceptor systems that enable such dynamics. As an example, upconversion reactions can occur when states of acceptor quantum systems (such as nuclei e.g. Pd nuclei) are resonant with coupled donor quantum systems (such as deuteron pairs as in D₂) and when coherence is maintained long enough and over a large enough coherence domain/coherence length that within the coherence time several donor quantum systems collectively/cooperatively upconvert, leading to a transfer of excitation from several donor quantum systems to fewer acceptor quantum systems. An example is the transfer of multiple 24 MeV quanta from |D₂> to sufficiently long-lived rotational nuclear states of lattice nuclei, several of which collectively upconvert to cause symmetric or close-to-symmetric fission of lattice nuclei such as Pd. Effectively, this represents a form of fission induced by a preceding fusion reaction in combination with quantum coherent dynamics. Consider, for instance, two |D₂>→|He-4> de-excitations that drive a |Pd>→|Pd*> excitation on the order of 48 MeV with subsequent disintegration of the |Pd*> state, or further transfer according to the same principles. Similarly, in another system, a dominant pathway could involve multiple |D₂>→|He-4> de-excitations with simultaneous excitation of a |Pd>→|Pd*> state beyond 48 MeV with the ultimate result of symmetric or close-to-symmetric Pd fission products. In the case of Pd nuclei, if several tens of MeV of excitation are transferred to such acceptor quantum systems, fission (asymmetric or symmetric) results whereas the exact fission products depend on the amount of energy received by the acceptor before fission taking place (and thus breaking the coherence).

Downconversion: A process analogous to upconversion, but reverse compared to above-described upconversion (where the state energy from two or more quantum systems in a coherent ensemble is transferred to a single quantum system), is dowconversion (where the state energy from one quantum system in a coherent ensemble is transferred to two or more quantum systems). In the case of nuclei as quantum systems of interest, this is advantageous if the thus induced behavior of the multiple acceptor nuclei (excited by the simultaneous de-excitation of a single nuclear quantum system such as |D₂>) are desirable e.g. if their resulting particle emission or further downconversion (in the daisy chain) exhibits desirable physical outcomes.

Energy upconversion and downconversion during nonradiative energy transfer—also known as quantum pooling and quantum cutting in some contexts—can be thought of as somewhat analogous to an electric transformer that, for instance, converts high voltage at low current to low voltage at high current (whereas total amounts of power and energy are conserved of course). Similarly, a downconversion process can involve a small number of high-energy quanta that gets converted (via transfer to sufficiently resonant receiver systems) to a large number of low-energy quanta. In one embodiment, the method and system presented here is used to optimize the performance of such transformer-like systems i.e. systems centered around and drawing on downconversion and upconversion behavior.

Energy amplification: The above-discussed case of U-238 disintegration via alpha emission induced by a |D₂>→|He-4> state transition is an example of what can be called nonradiatively induced energy amplification, as the initial reaction that would conventionally expected to yield a released energy quantum of 24 MeV in a device with a coupled quantum system where the |D₂>→|He-4> transition strongly couples to a U-238 nucleus (that subsequently disintegrates) yields alphas of 28 MeV. Therefore, the overall yield of such a system is effectively amplified by the deliberate and well-informed (per the steps described according to this invention) composition, configuration, and stimulation of the sample.

### Broad Use Cases

Use cases for the devices designed and optimized according to this invention can be grouped into different areas of application such as heat production for industrial purposes or electricity production for remote sensors, energy storage in metastable excited states at the atomic and nuclear level i.e. quantum batteries, and information storage and processing at the atomic and nuclear level i.e. quits, each of which come with their own sets of requirements and constraints, based on which an optimization process needs to be applied to arrive at a suitable and competitive configuration and implementation. Use cases can be much more specific within broad categories and with highly specific and context-dependent requirements and constraints that then drive optimization and device design processes.

The principles of optimizing the performance of coupled quantum systems and their dynamics according to this invention apply to all of these systems, albeit with differing output parameters to be optimized for and different sets of input parameters to be considered in the design process. For instance, in quantum batteries, the excited states of quantum systems (atomic level or nuclear level) are used to temporarily store energy and retrieve such energy upon demand. Transfer of energy into and out of the excited states of such quantum systems occurs through temporarily enhanced couplings that cause the desired dynamics. The development of designs suitable for large-scale dissemination requires the optimization of this process, based on an optimal set of input parameters such as the composition of materials used, the environmental/boundary conditions, and the stimulation characteristics. Such an optimization follows the same general process described this invention.

Individual broad use cases are spelled out and discussed in more detail below. Note that this list of use cases is exemplary and not exhaustive. Note also that use cases can be more fine-grained and differentiated, e.g. the broad use case nuclear-to-electric conversion can include many specific, context-dependent use cases (such as, as examples, nuclear-to-electric conversion for grid scale electricity production and nuclear-to-electric conversion for remote sensor electricity production). Each of the broad classes of use cases and each of the specific use cases has their own sets of requirements and constraints and their own versions of dominant designs for the particular application—therefore requiring a dedicated optimization and design process according to this invention.

Suppression of neutron emission: A use case is the modification of nuclear reactions towards the avoidance of neutron emission. The goal here is to transfer energy away from certain excited states in the sample, before their decay, that would otherwise be expected to result in neutron emission. What is needed to that end are receiver quantum systems that can be coupled to—sufficiently strongly for such a transfer to occur fast enough to prevent neutron emission—, and for the subsequent de-excitation to occur through channels that do not include neutron emission (e.g. charged particle emission only or mainly).

Nuclear-to-electric conversion: It can be desirable to turn released nuclear binding energy (that results from nuclear reactions or from the decay from higher nuclear excited states) into electric current. A wide range of technologies have been described in the literature for the conversion of kinetic energy of charged particles to electricity. Examples are so-called Venetian Blinds and alphavoltaic devices. Many of the proposed conversion devices function best if the available energy is rather predictable and within a particular energy range. This invention can aide in matching the form of energy provided by a generating device to the requirements of a conversion device, thereby increasing the efficiency of such a conversion process. Specifically, the generation of charged particles of specific characteristics can be desirable. Certain types of charged particles and certain energy ranges of these particles may be particularly desired, as a particular electric conversion system is laid out or optimized for them. This invention can be employed to convert outputs from nuclear processes in such a way that specific output parameter spaces are reached that are desired for follow-on processes such as electric conversion processes. To design a corresponding system, a practitioner identifies from the chart of nuclides those nuclides with energy levels resonant to the preceding driving reaction and with subsequent decay channels that result in the desired output reaction products. Driving the output product generation can also involve downconversion, upconversion, and/or multiple preceding energy transfer steps to arrive at the best combination of a driving reaction (that releases energy into the dynamic system) and output product generating reaction. In case of intermediate steps, intermediate acceptor nuclei need to be considered. Eventually the excitation energy will be received by the final acceptor nuclei which will then undergo de-excitation from the received excited state resulting in decay products that can then be used for electric conversion, tailored to the chosen energy conversion process.

Nuclear-to-heat conversion: In other applications according to this invention, nuclear excitation energy may be preferred to be converted to end products different from the ones mentioned above. Instead, nuclear excitation energy may be preferred to be converted into what manifests macroscopically as heat. In this case, the sequencing of nuclear state transitions—where the different steps of the sequence are connected via nuclear excitation transfer, as described—is employed in a way that at the end of the sequence (even if it is a short sequence e.g. consisting of only one transfer) are excited phonon modes as the final acceptors of nuclear excitation (i.e. excitation that drives the dynamics and that originated as released and converted nuclear binding energy). The conversion from nuclear excitation energy to phonon excitation energy may be aided by introducing “stepping stones” i.e. intermediate acceptor nuclei that enable a sequence of nuclear excitation transfers that convert a large quantum of energy in the form of nuclear excitation (that results from an earlier nuclear reaction) into multiple smaller quanta of nuclear excitation by transferring the large quantum of energy to multiple acceptor nuclei (with corresponding energy levels that collectively allow for the acceptance of the large quantum of energy). In a sequence of nuclear conversion/nuclear excitation transfers, these acceptor nuclei then can act as donor nuclei as the energy gets further transferred to another group of acceptor nuclei (and possibly further divided into smaller energy quanta in the process). This can be repeated multiple times in order to efficiently convert to small enough energy quanta such that excited phonon modes can serve as eventual acceptors of the energy in this sequence of excitation transfers. Modeling and optimizing such processes ought to take into account feedback loops that results from further populated phonon modes via downconversion: enhanced phonon modes can mean enhanced phonon-nuclear coupling and thus enhanced energy transfer dynamics. When conducting the optimization process, and corresponding device design, the equilibrium state after such a feedback process plays out ought to be considered (to the extent possible).

Quantum batteries: Particularly relevant for quantum batteries, and especially for nuclear quantum batteries, are the processes of upconversion and downconversion as designed and optimized according to this invention (see above and below for a more detailed elaboration as well as examples for corresponding simulations). A key issue for quantum systems used for energy storage via metastable quantum states, i.e. quantum batteries, is how to move energy into and out of such states. This is complicated by the fact that there is often an energy mismatch between the state transitions in the quantum systems that are meant to hold the energy (e.g. nuclear states at the keV or MeV level) and the way in which energy is initially available and in which the energy is eventually needed again (e.g. electrons, ions, or photons at the eV level). This mismatch can be bridged if the excitation and de-excitation of quantum systems used as quantum batteries is conducted in combination with upconversion and downconversion processes e.g. when multiple low-level oscillators (e.g. eV level) get pooled toward the coupling-induced excitation of a single—or multiple—higher level quantum system (e.g. keV level). And, similarly, when the coupling-induced de-excitation of a single keV level quantum system results in the transfer of energy to multiple eV level oscillators. The performance of such charging and discharging processes can be modeled and simulated—and then optimized through iterations as described—by the method and system described here. This in turn informs and drives the design of high-performing devices for this use case category.

Nuclear qubits: When quantum systems are used for information storage (whereas the storage of information takes place through the storage of energy in quantum systems even if that amount of energy is small), changing the information content is an important functionality of the overall device. The simulation and optimization processes described here also apply to the optimization of qubit systems, including those that use nuclei as qubits i.e. nuclear qubits, where excitation and de-excitation is sought to be conducted in a controlled manner and where the overall performance of the system is sought to be optimized based on given criteria and constraints (e.g. cost, operating conditions, reliability etc.). In one exemplary embodiment, phonons are used to enhance couplings between the states of quantum systems such as nearby nuclei in order to flip qubits i.e. change the information content of such quantum systems through deliberate excitation or de-excitation. Phonons can be used to that end in analogous ways to the phonon-mediated controlled excitation and de-excitation of quantum systems in energy-minded applications (as described above and below), according to this invention.

Exemplary Embodiment of a Device Design as Designed and Optimized According to this Invention

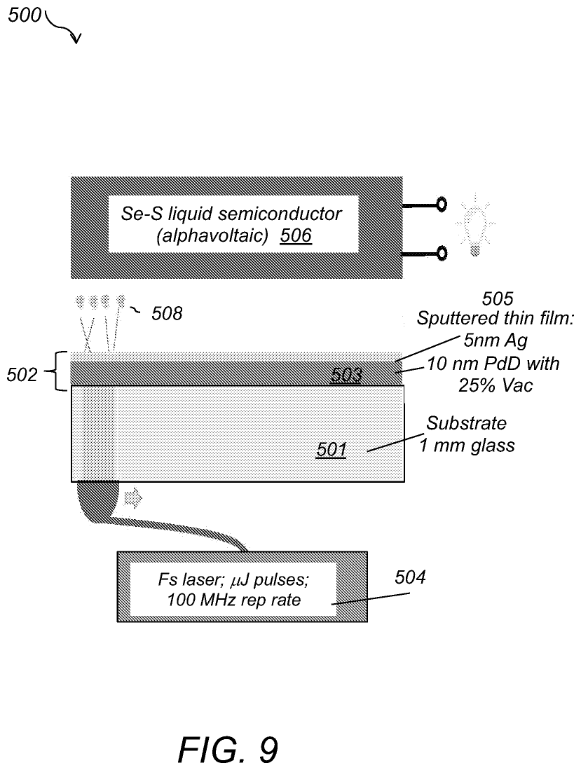

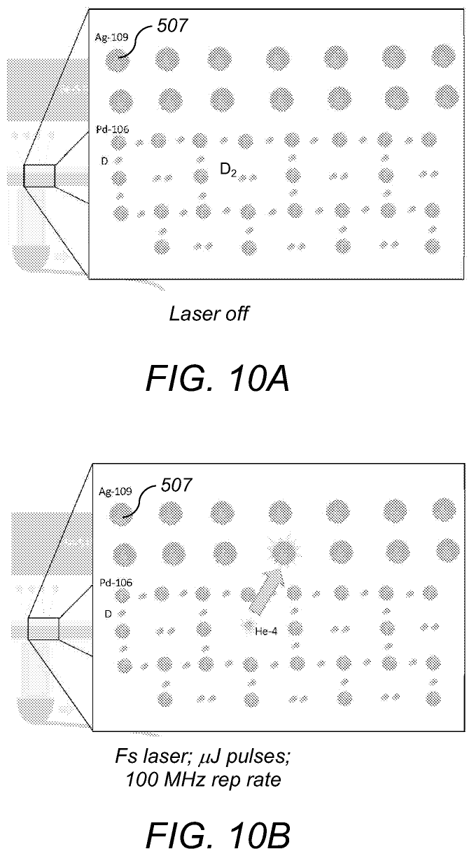

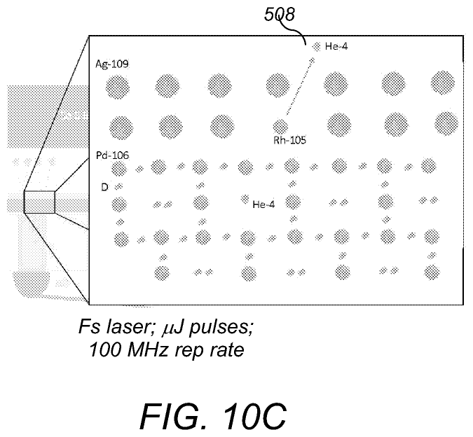

As described above, the optimization and design principles—and the underlying modeling and simulation techniques—according to this invention apply to a wide range of applications. Referring to FIG. 9 to FIG. 10C, an exemplary embodiment for a device to be designed and optimized according to this invention is a power conversion device 500. Power conversion device 500 includes a sample material 502 that is deposited on a substrate 501. In this example, the sample material 502 is a composite of thin films, comprising a 5 nm Ag-109 layer 505 and a 10 nm Pd-106 layer 503 over an area of 5 mm×20 mm. The Pd thin film 503 is deposited on a 1 mm glass substrate 501 in a mixed Ar and D atmosphere, leading to a high concentration of vacancies with deuterium interstitials. A vacancy content of at least 1% is sought, ideally 10% and more. The Ag layer 505 is added based on well-established thin film deposition techniques. Leaking of deuterium from the sample material is prevented by coating a thin passivation layer on the sample surface where leaking may occur, or by freezing the sample through a thermoelectric cooler. Stimulation is provided by a laser 504 that provides coherent photons that couple with the sample lattice, generating coherent plasmons and phonons in the sample as a result. The coupling between coherent oscillators in the sample 502 and nuclear states leads to a modification of state transitions. As described above, this modification of state transition characteristics can be modeled as a function of laser and stimulation characteristics, taking into account collective quantum effects such as superradiance as a function of the size of the coherence domain (i.e. the size of the coupled quantum system) and feedback loops such as oscillator modes getting pumped and thus enhanced by downconversion of state transition quanta (and the corresponding coupling strengths enhanced). In this embodiment, Ag-109 nuclei 507 are used as receiver quantum systems that disintegrate upon receipt of transferred excitation from a D₂>→|He-4> transition (FIG. 10A to FIG. 10B), whereas the excitation transfer occurs as a result of temporarily enhanced couplings between pairs of deuterium nuclei in the lattice as donor systems and resonant or close-to-resonant receiver systems. The disintegration of receiver nuclei 507 results in charged particle emission 508 which in this embodiment is captured via an alphavoltaic device 506 that is attached across the layer of receiver nuclei i.e. the Ag-109 nuclei. The alphavoltaic device 506 turns the kinetic energy of charged particles 508 into an electric current that can be used to operate electric devices or can be fed into a power grid. In this embodiment, the alphavoltaic device 506 is a liquid-selenium based Se-S semiconductor which is attached on top of the Ag-109 layer in close proximity of <1 mm. The receiver layer 502 can be doped with further nuclei of a different species to achieve a more desirable energy range of resulting charged particles that is better matched to the peak performance of the alphavoltaic device 506. Output power of the system can be adjusted by controlling which region of the sample is stimulated at a given point in time. In one embodiment, the laser spot size is 100 microns in diameter and the position where the laser light interacts with the sample is adjusted mechanically (by moving the sample or by moving the laser spot). As the flux of charged particles 508 resulting from reactions in the sample—and therefore overall performance of the device—decreases, the laser spot is moved relative to the sample to a neighboring region of the sample that has not yet participated in the reactions. In another exemplary embodiment, the receiver nuclei are not provided through a separate thin-film layer but are directly embedded in the lattice with the donor systems e.g. via implantation or alloying. In another exemplary embodiment, the laser as a source for the production of phonons in the sample is replaced by an electrical means of coherent phonon production in the sample. In this case, a device of the type described above can be implemented without moving parts in a solid-state format i.e. in a wafer-like form factor. Fabrication of the latter draws on microfabrication techniques used for other highly integrated and nanostructured products such as in MEMS, semiconductor or integrated circuit design and production.

Optimizations of the performance of the above systems include maximizing power output, and in view of maximizing power output maximizing longevity of the system, and/or minimizing cost. According to the simulation and optimization processes of this invention, such output variables can be derived as functions of input variables of the system, including the composition of the lattice, the environmental/boundary conditions, and the stimulation characteristics. Through the modeling process, parameters of the system can then be optimized and tested in practice. Tests in practice are fed back to the modeling process to make adjustments and refinements to the modeling process.

In another exemplary embodiment of the device described above, different architectural components of the device are arranged across different zones of the device.

Specifically, in a power production device, the components—or zones—include a fuel storage component where fuel to be used in nuclear reactions is stored such as atoms of hydrogen or one of its isotopes. Either in the same or in a separate component, the nuclear reactions take place in a controlled or semi-controlled manner. If the latter is a separate component, then transport is arranged of the fuel atoms to the nuclear reaction component of the device (e.g. through diffusion). In the component where the nuclear reaction takes place, excitation transfer is facilitated based on the above described principles. Acceptor nuclei of a desired nuclear species are arranged near the donor nuclei which initially hold nuclear excitation released from the nuclear reaction. Mediated by common oscillator modes, such as common phonon modes, this nuclear excitation transfers to acceptor nuclei, triggering a secondary nuclear reaction chosen for its desirable reaction products. The latter can comprise charged particles of a desired kind/energy level. In an additional component of the overall system, these charged particles are then converted to electric current based on known conversion principles described in the literature. A solid-state form factor of such a system—i.e. built into a semiconductor chip like packaging—represents an important feature as it can be designed in a compact, low-maintenance way and mass produced using common principles of mass production as used for similar devices.

Illustration of Modeling Principles for Coupled Systems with Excitation and De-Excitation Dynamics

The basic mechanism of excitation transfer in coupled systems—and some of the key modalities such as upconversion and downconversion, as described above—can be illustrated and modelled through simple yet powerful approaches that draw on classical analogs to quantum-level excitation transfer in coupled quantum systems. Authors such as Briggs & Eisfeld (2012) have shown the direct transferability and applicability of classical analogs to quantum models when it comes to modeling the dynamics that result from coupled states, including in some instances the quantitative agreement of predicted behavior in classical and quantum models. Therefore, insights gained and derived from the models presented in this section are directly applicable to the design of systems of quantum-coherent nuclear engineering i.e. systems designed to trigger and optimize specific nuclear reactions with desirable outputs as a result of the system composition and stimulation (such as coherent phonons or coherent photons).

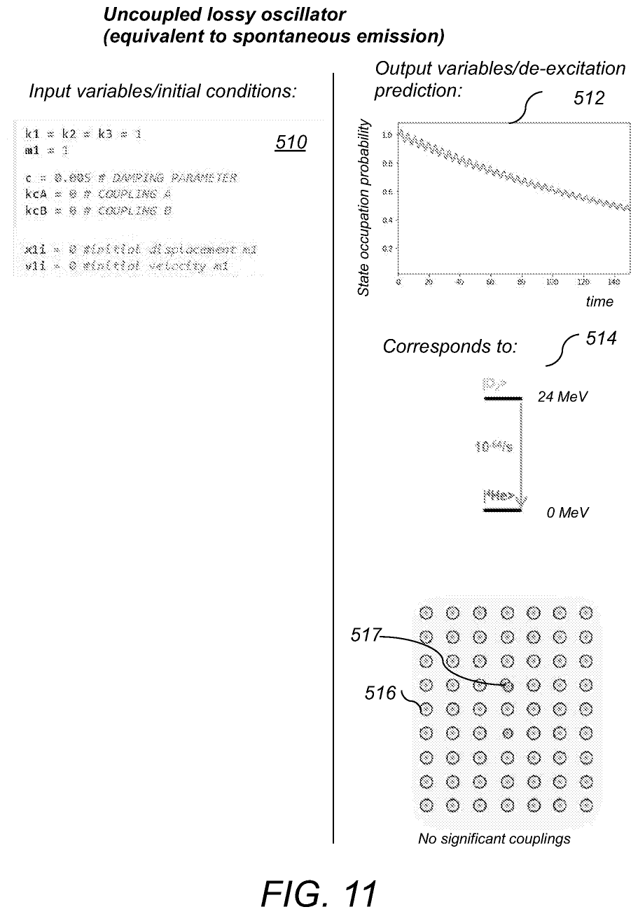

The basic mechanisms of coupling-induced i.e. nonradiative excitation transfer are described below with reference to FIG. 11-FIG. 15. Here quantum systems are represented as oscillators (akin to pendula) and the couplings between them are represented as connecting springs with a spring constant that corresponds to the coupling strength. Resonance is given if coupled oscillators exhibit the same intrinsic frequencies.

To illustrate key principles underlying the modeling process, examples with one to three oscillators (representing quantum systems to be coupled) are described herein. The dynamics of energy transfer between three oscillators 1, 2 and 3 that are coupled via couplings with coupling strengths kcA and kcB can be derived from basic force equations, as will be described below. From these force equations differential equations are derived that express the positions and velocities of all oscillators at any point in time—and thus the energy held by each oscillator at any point in time. The time evolution of these values—and thus the overall behavior of this system—is calculated once initial values are considered. For this part of the modeling process, initial values such as initial energy levels occupied by each oscillator and coupling strengths between them represent input variables and state transition rates represent output values. When extended to represent actual conditions in a lattice sample and to capture quantum effects such as superradiance, comparable models form part of Step D. The input variables and output variables of Step D represent intermediate variables in the overall simulation and optimization process.

For three coupled oscillators the force equations are as follows:

$${F}_{1} = m {\ddot{x}}_{1} = - {k}_{1} {x}_{1} - {k}_{c} ( {x}_{1} - {x}_{2} ) - c {\dot{x}}_{1}$$

$${F}_{2} = m {\ddot{x}}_{2} = - {k}_{2} {x}_{2} - {k}_{c} ( {x}_{2} - {x}_{1} ) - {k}_{c} ( {x}_{2} - {x}_{3} ) - \dot{{cx}_{2}}$$

$${F}_{3} = m {\ddot{x}}_{2} = - {k}_{3} {x}_{3} - {k}_{c} ( {x}_{3} - {x}_{2} ) - \dot{{cx}_{3}}$$

$$\ddot{x} + c \dot{x} + \frac{k}{m} x = 0$$

where k₁, k₂, k₃ are the spring constants of the three oscillators, k_{c} is the coupling constant between the oscillators (here: kc=kcA=kcB), m is the mass of the oscillators, x₁, x₂, x₃ are the displacements of each oscillator, the single dotted x₁, x₂, x₃ are the velocities and the double dotted x₁, x₂, x₃ are the accelerations.

Organizing the force equations in terms of x₁, x₂ and x₃ results in the following differential equations that describe the time evolution of the coupled system:

$${\ddot{x}}_{1} = {\dot{υ}}_{1} = {x}_{1} \frac{- {k}_{1} - {k}_{c}}{{m}_{1}} + {x}_{2} \frac{{k}_{c}}{{m}_{1}} - c {υ}_{1}$$

$${\ddot{x}}_{2} = {\dot{υ}}_{2} = {x}_{1} \frac{{k}_{c}}{{m}_{2}} + {x}_{2} \frac{- {k}_{2} - 2 {k}_{c}}{{m}_{2}} + {x}_{3} \frac{{k}_{c}}{{m}_{2}} - c {υ}_{2}$$

$${\ddot{x}}_{3} = {\dot{υ}}_{3} = {x}_{2} \frac{{k}_{c}}{{m}_{3}} + {x}_{3} \frac{- {k}_{3} - {k}_{c}}{{m}_{3}} - c {υ}_{3}$$

FIG. 11 shows a single oscillator without any strong couplings to other oscillators. The oscillator decays as it couples very weakly to a large number of modes in the environment i.e. it is lossy. The input variables 510 for the uncoupled damped oscillator include spring constants k1=k2=k3=1, coupling parameters k_{cA}=k_{cB}=0, damping parameter c=0.005, oscillator mass m₁=1, initial displacement xli=0 and initial velocity vli=0. The calculated output variables 512 include the plot of the state occupancy probability versus time. The decay of the oscillator 512 then follows an exponential trajectory with a decay factor c. Again, the behavior of this oscillator decaying is equivalent to a quantum system de-exciting where the total energy of the oscillator corresponds to the state occupation probability of the quantum system. Such a decay corresponds to what is known as spontaneous emission. In the realm of quantum-coherent nuclear engineering, this behavior corresponds to the natural decay of a nuclear excited state, as is the case with the spontaneous D₂→He-4 reaction in isolation (see Koonin & Nauenberg 1989). An example is a deuteron pair 517 embedded in a Pd lattice 516 without any significant couplings to nuclear states of other quantum systems.

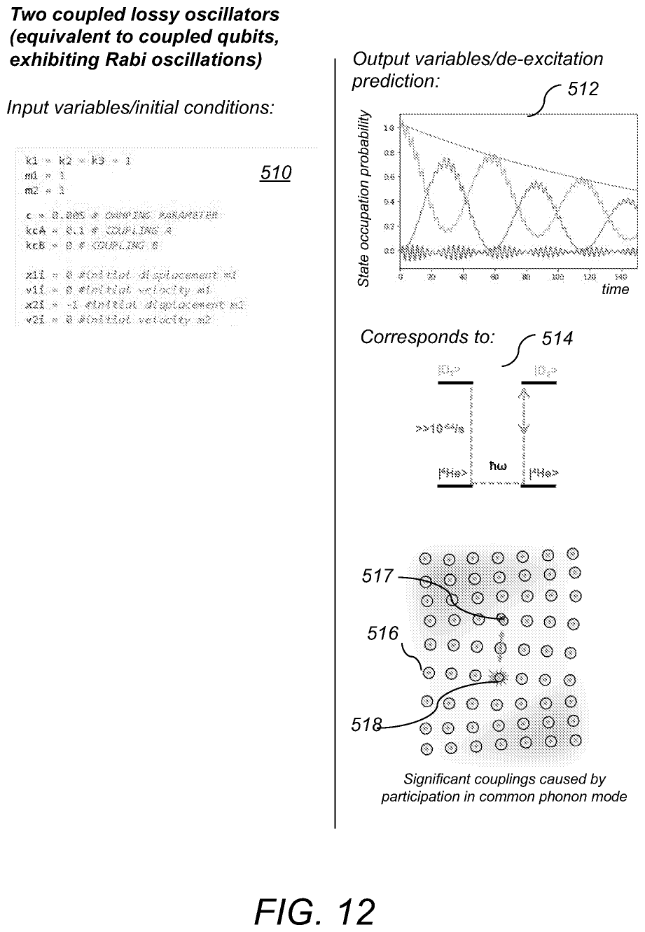

FIG. 12 shows an oscillator 1 that is strongly coupled to another oscillator, oscillator 2. Whereas the loss factor i.e. the decay factor c is equivalent to that of the previous example, the actual de-excitation of oscillator 1 is much accelerated compared to the previous example. This acceleration of de-excitation grows further with the increase of the coupling strength between the resonant oscillators (here: kcA). From a quantum dynamics perspective, the behavior exhibited in this example is known as Rabi oscillations. In the realm of quantum-coherent nuclear engineering, this behavior corresponds to, for example, an accelerated de-excitation of the D₂>→|He-4> transition along with a corresponding |He-4>→D₂> transition in a strongly coupled system. Note that this particular configuration can be considered as a closed quantum system where energy is redistributed within the closed system without a strong pathway to escape the closed system. An example is a deuteron pair 517 embedded in a Pd lattice 516 with a nearby He-4 nucleus 518 and with both the deuteron pair and the He-4 nucleus participating in the same coherent oscillator mode such as a phonon mode. If the coupling strength becomes large enough, Rabi oscillations can occur between the coupled deuteron pair and He-4 nucleus. Note that the simulation shows how the coupling can remain substantially lower in energy than the energy that gets transfer through the mediation of the coupling. Still, a higher coupling strength leads to faster oscillations and therefore to faster de-excitation of an initially excited system.

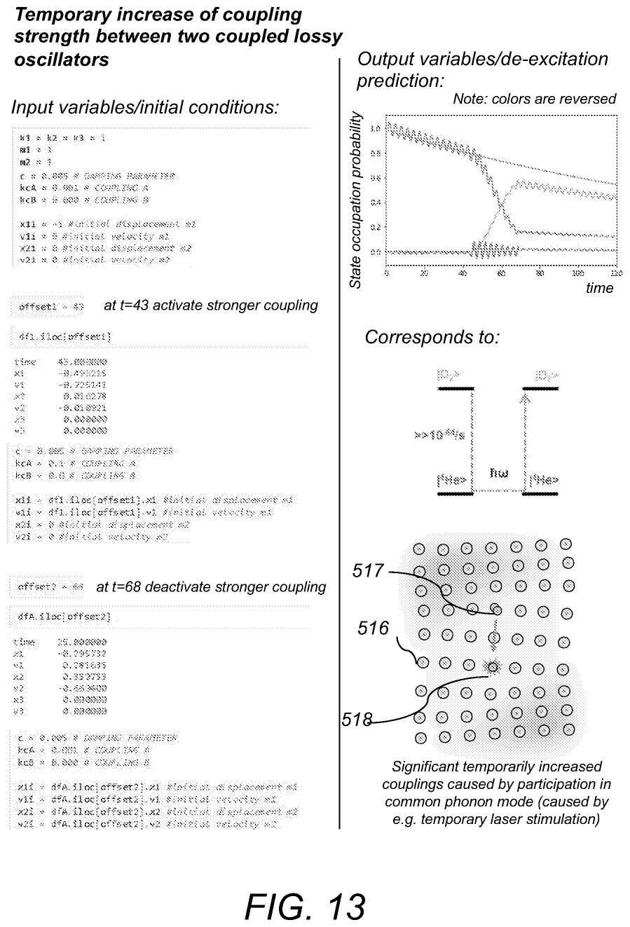

FIG. 13 shows simulation input and output variables for a scenario with two oscillators where the coupling between them is initially weak and then temporarily enhanced (from time t=43 to time t=68). During the period with larger coupling strength between the oscillators, energy (or, from another perspective, state occupation probability) gets transferred from oscillator 1 to oscillator 2. Such dynamics correspond to the so-called flipping of a qubit. The temporary enhancement can be caused, for instance, by laser stimulation that populates phonon modes or photon modes that are common to the quantum systems and interact with the states of the quantum systems. In the realm of quantum-coherent nuclear engineering, when considering a scenario of this sort for a coupled deuteron pair 517 and He-4 nucleus 518, the application potential is very limited, as from a macroscopic perspective the likely final state looks equivalent to the initial state (only with the deuteron pair and He-4 nucleus reversed). That is because the final state of the He-4 nucleus excited to a deuteron pair is as stable as the initial configuration. The situation is different if another receiver nucleus is made available and coupled to instead of the He-4 nucleus. If the receiver nucleus is, for instance, a U-238 nucleus which, due to its high density of states around 24 MeV, is also close-to-resonant to the D₂>→|He-4> transition, the initial dynamics are equivalent: the de-excitation of the |D₂>→|He-4> transition is accelerated along with the corresponding excitation of the U-238 nucleus to an excited state near 24 MeV (resonant or close-to-resonant excitation transfer). However, as most |U-238*> excited states near 24 MeV are highly unstable, the U-238* nucleus is expected to disintegrate. An incoherent disintegration breaks the coupled system and is therefore equivalent to reducing the coupling strength to insignificant levels, as seen in the example. Therefore, this simulation, and the underlying model, also describes in basic terms the corresponding reaction shown in FIG. 13.

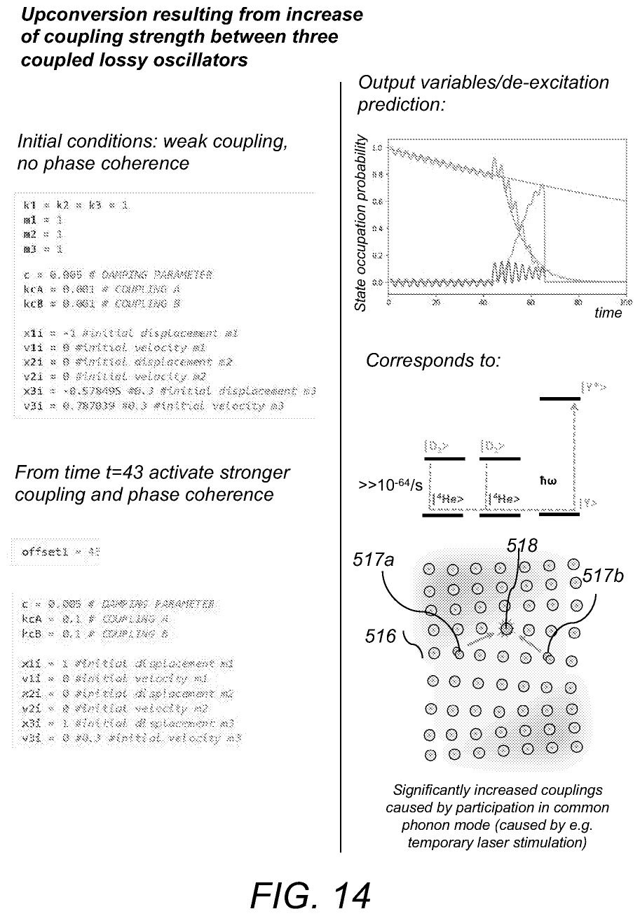

FIG. 14 shows simulation input and output variables for a scenario with three oscillators where the coupling between them is initially weak and then temporarily enhanced (at time t=43). Here, per the initial conditions i.e. the input variables to the simulation, two oscillators 517a, 517b are initially excited and one 518 is unexcited. Due to resonance between the oscillators, the two excited oscillators 517a, 517b collectively transfer their energy to the unexcited oscillator 518 once the coupling strength is increased. In the realm of quantum-coherent engineering, this behavior corresponds to a scenario where two deuteron pairs 517a, 517b, are embedded in a highly loaded part of lattice such as a Pd lattice (preferably in monovacancies) 516 which is then stimulated to create strong couplings between nuclear states e.g. by a laser, as described above. As two deuteron pairs strongly couple to a single Pd nucleus, both of the D₂>→|He-4> transition can be accelerated with the corresponding excitation of the Pd nucleus to an |Pd*> excited state near 48 MeV (2×24 MeV). The highly excited Pd* nucleus would be expected to subsequently disintegrate—unless another excitation transfer happens at such a fast rate that it would preclude incoherent disintegration. It can be seen that if more quantum systems participate in a simultaneous upconversion process, or if energy transfer is fast enough that a receiver nucleus can receive excitation coherently repeatedly before disintegration, then very high excitations of receiver nuclei can be achieved. In nuclei such as the nuclei of Pd isotopes, this translates into disintegration in the form of increasingly more symmetric fission per the known literature on symmetric and asymmetric fission (the more symmetric the higher the excitation that causes disintegration).

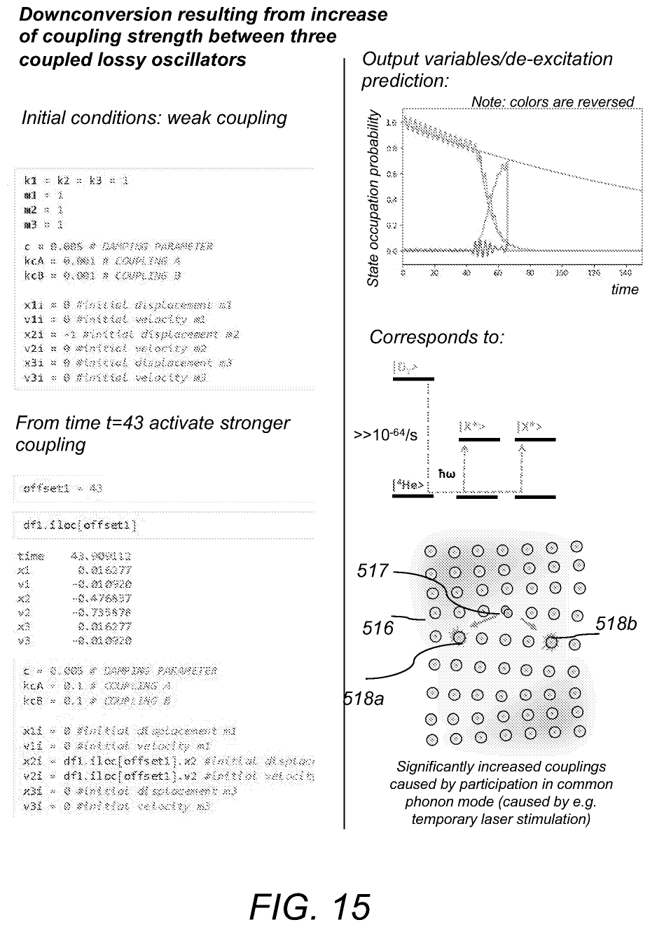

FIG. 15 shows simulation input and output variables for a scenario with three oscillators where the coupling between them is initially weak and then temporarily enhanced. Here, per the initial conditions i.e. the input variables to the simulation, one oscillator 517 is initially excited and two oscillators 518a, 518b are initially unexcited. Due to resonance between the oscillators, the excited oscillator 517 transfers its energy in a distributed manner to the two unexcited oscillators 518a, 518b once the coupling strength is increased. In the realm of quantum-coherent engineering, this behavior corresponds to a scenario where a deuteron pair 517 is embedded in a highly loaded part of lattice 516 such as a Pd lattice (preferably in monovacancies) which is then stimulated to create strong couplings between nuclear states e.g. by a laser. As a deuteron pair strongly couples to two resonant lattice nuclei 518a, 518b, the D₂>→|He-4> transition can be accelerated with the corresponding excitation of the two lattice nuclei (here referred to as Y) near 12 MeV each (0.5×24 MeV). Such lattice nuclei would then either decay incoherently or act as donor systems in further excitation transfer (and potentially a further downconversion step) if coherent excitation transfer occurs faster than incoherent decay. State lifetimes before incoherent decay can be looked up from nuclear data databases or simulated from nuclear codes such as nuclear shell model codes. Follow-on excitation transfer rates can be predicted based on the coupling strengths, the degrees of resonance between the coupled systems, and superradiant enhancement of the transfer dynamics which depends on the size of the coupled systems.

Note that the general environment of the downconversion example and the upconversion example is similar—i.e. deuteron pairs embedded in a highly D-loaded Pd lattice and stimulated by a laser. What dynamics ensue in practice depends critically on what the vicinity looks like of the deuteron pairs that form part of the coupled quantum systems that result from the stimulation. If the dominant resonances in the coupled quantum system under consideration, at the coupling strengths that result from the stimulation, are ones associated with downconversion, then downconversion will be dominant. Similarly, if the dominant resonances in the coupled quantum system under consideration, at the coupling strengths that result from the stimulation, are ones associated with upconversion, then upconversion with dominant. This further illustrates the power of the designer to affect dominant reactions in the sample by adjusting sample composition and configuration as well as stimulation characteristics—and the importance of the modeling, simulation, and optimization method described here to design devices with high-performance outcomes. Dominant outcomes for given samples and sample variants can be predicted based on the principles described here with further customization and extension to match the conditions of specific samples along with the specific requirements and constraints for the use case at hand.

What the examples above also show is that the fundamental nonradiative transfer dynamics are agnostic to the physical origin of the coupling that causes them—as long as the coupling strengths involved are large enough for such dynamics to occur. As a consequence, concrete values for coupling strengths to be used in the simulation process can be determined either from first principles or from experimental tests.

Note that in the figures accompanying the examples above (FIG. 11 to FIG. 15), for clarity and readability the lattice environment is shown in a simplified manner. In actuality, a larger number of participating quantum systems are needed in the coupled quantum system to achieve substantial enhancements from superradiance. In actuality, also more interstitials are needed to achieve sufficient stabilization of vacancies and di- deuterium formation (close proximity between interstitials).

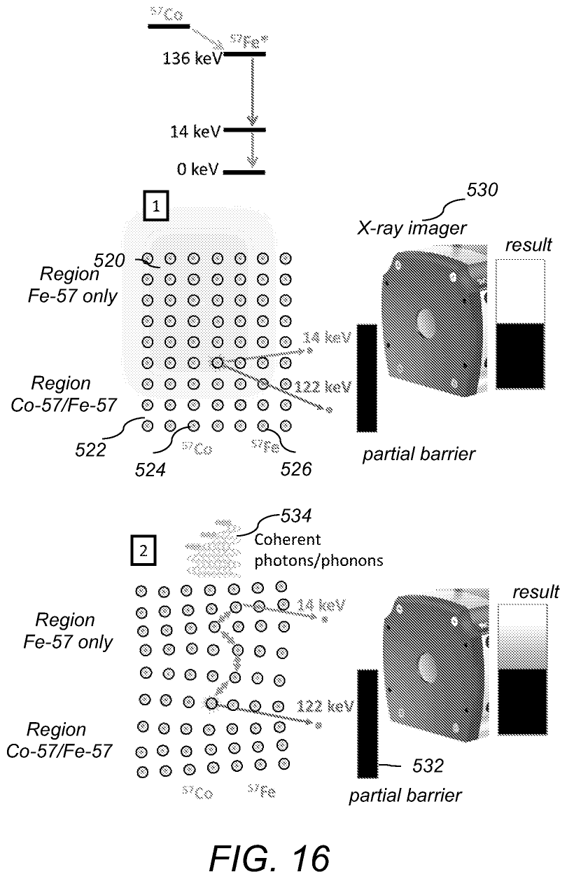

A configuration for the experimental determination of couplings strengths is shown in FIG. 16. In one exemplary embodiment, the effective coupling strength is determined between Fe-57 nuclei in a sample, as that coupling strength results from laser stimulation. The sample used in this example is a single-crystal with two regions: one region 520 with only Fe-57 nuclei 526 and one region 522 with both Fe-57 nuclei 526 and Co-57 nuclei 524 (here shown with a 1:1 ratio). Photon emission from the sample at the 14 keV range is monitored via an X-ray imaging device 530 such as the Andor ikon M X-ray camera. The Co-57/Fe-57 region 522 of the sample is then blocked with a barrier 532 such that the camera 530 observers the edge of the two regions without receiving/detecting any photons. The sample is then stimulated with laser photons 534 such as to generate coherent phonons in the sample of different energy levels (across the range from 10 MHz to 5 THz). For each phonon energy level, the delocalization of photon emission from the sample into the Fe-57 only region 520 is recorded via the X-ray imager. Delocalization occurs as a result of repeated excitation transfers across the coupled quantum systems that result from enhanced coupling strengths, caused by the laser stimulation. From the extent of delocalization at each phonon energy level, the coupling strength between the 14 keV states of coupled Fe-57 nuclei can be determined as a function of phonon energy.

The distance covered by transferred excitation within the decay time of the nuclear state is indicative of the transfer rate, and by extension of the coupling strength driving the transfers. For instance, observing a 1 mm delocalization into the Fe-57 only region 520 that results from repeated transfer across coupled systems of a size of about 10 nm each (coherence domain resulting from the stimulation) suggests about 100,000 transfers. When observed in a Fe-57 sample, at the 14 keV line, then we also know the decay time of that state which is about 100 ns (98 ns to be precise). This corresponds to a transfer rate of about 1/fs (i.e. about 1 THz). This, in turn, corresponds to an effective coupling strength (after all enhancements) of about 1 meV. Thus obtained coupling strengths can be used, for comparable contexts, in modeling and simulation processes as described herein. They can also be compared to first principles calculations of various coupling strengths, as described above.

The same principles and considerations, slightly adjusted to the particular circumstances e.g. by measuring emitted alphas instead of photons, can be applied to experimentally determining coupling strengths between other nuclear states as well as other stimulation characteristics, stimulation techniques and settings. The approach is also suitable for testing candidate sample configurations such as particular sample compositions.

One embodiment of the invention is used to compare the data obtained through simulation with the data from experimentally tested materials. One embodiment of the invention is used to design new experiments or devices based on insights from simulations. Experiments can then be adjusted as a result of simulations. Simulations can then be adjusted as a result of experiments and device performance.

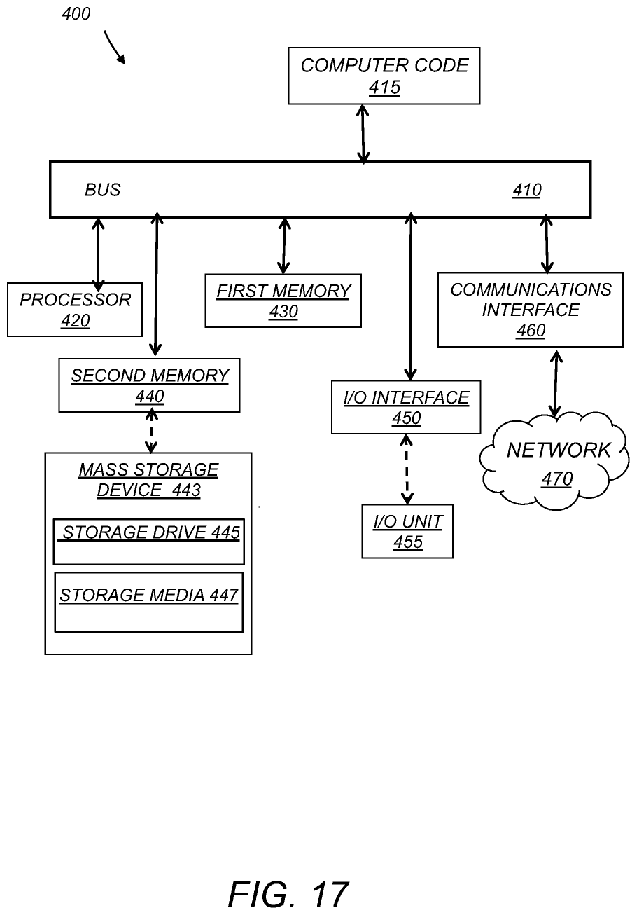

Referring to FIG. 17, an exemplary computer system 400 or network architecture that may be used to implement the system of FIG. 5 and the method of FIG. 6A of the present invention includes a processor 420, first memory 430, second memory 440, I/O interface 450 and communications interface 460. All these computer components are connected via a bus 410. One or more processors 420 may be used. Processor 420 may be a special-purpose or a general-purpose processor. As shown in FIG. 17, bus 410 connects the processor 420 to various other components of the computer system 400. Bus 410 may also connect processor 420 to other components (not shown) such as, sensors, and servomechanisms. Bus 410 may also connect the processor 420 to other computer systems. Processor 420 can receive computer code 415 via the bus 410. The term “computer code” includes applications, programs, instructions, signals, and/or data, among others. Processor 420 executes the computer code 415 and may further send the computer code via the bus 410 to other computer systems. One or more computer systems 40 may be used to carry out the computer executable instructions of this invention.

Computer system 400 may further include one or more memories, such as first memory 430 and second memory 440. First memory 430, second memory 440, or a combination thereof function as a computer usable storage medium to store and/or access computer code. The first memory 430 and second memory 440 may be random access memory (RAM), read-only memory (ROM), a mass storage device, or any combination thereof.

As shown in FIG. 17, one embodiment of second memory 440 is a mass storage device 443. The mass storage device 443 includes storage drive 445 and storage media 447. Storage media 447 may or may not be removable from the storage drive 445. Mass storage devices 443 with storage media 447 that are removable, otherwise referred to as removable storage media, allow computer code to be transferred to and/or from the computer system 400. Mass storage device 443 may be a Compact Disc Memory, ZIP storage device, tape storage device, magnetic storage device, optical storage device, Micro-Electro-Mechanical Systems (“MEMS”), nanotechnological storage device, floppy storage device, hard disk device, USB drive, among others. Mass storage device 443 may also be program cartridges and cartridge interfaces, removable memory chips (such as an EPROM, or PROM) and associated sockets.

The computer system 400 may further include other means for computer code to be loaded into or removed from the computer system 400, such as the input/output (“I/O”) interface 450 and/or communications interface 460. Both the I/O interface 450 and the communications interface 460 allow computer code to be transferred between the computer system 400 and external devices or webservers including other computer systems. This transfer may be bi-directional or omni-direction to or from the computer system 400. Computer code transferred by the I/O interface 450 and the communications interface 460 are typically in the form of signals, which may be electronic, electromagnetic, optical, or other signals capable of being sent and/or received by the interfaces. These signals may be transmitted via a variety of modes including wire or cable, fiber optics, a phone line, a cellular phone link, infrared (“IR”), and radio frequency (“RF”) link, among others.

The I/O interface 450 may be any connection, wired or wireless, that allows the transfer of computer code. In one example, I/O interface 450 includes an analog or digital audio connection, digital video interface (“DVI”), video graphics adapter (“VGA”), musical instrument digital interface (“MIDI”), parallel connection, PS/2 connection, serial connection, universal serial bus connection (“USB”), IEEE1394 connection, PCMCIA slot and card, among others. In certain embodiments the I/O interface connects to an I/O unit 455 such as a user interface, monitor, speaker, printer, touch screen display, among others. Communications interface 460 may also be used to transfer computer code to computer system 400. Communication interfaces include a modem, network interface (such as an Ethernet card), wired or wireless systems (such as Wi-Fi, Bluetooth, and IR), local area networks, wide area networks, and intranets, among others.

The invention is also directed to computer products, otherwise referred to as computer program products, to provide software that includes computer code to the computer system 400. Processor 420 executes the computer code in order to implement the methods of the present invention. In one example, the methods according to the present invention may be implemented using software that includes the computer code that is loaded into the computer system 400 using a memory 430, 440 such as the mass storage drive 443, or through an I/O interface 450, communications interface 460, or any other interface with the computer system 400. The computer code in conjunction with the computer system 400 may perform any one of, or any combination of, the steps of any of the methods presented herein. The methods according to the present invention may be also performed automatically or may be invoked by some form of manual intervention. The computer system 400, or network architecture, of FIG. 17 is provided only for purposes of illustration, such that the present invention is not limited to this specific embodiment.

Several embodiments of the present invention have been described. Nevertheless, it will be understood that various modifications may be made without departing from the spirit and scope of the invention. Accordingly, other embodiments are within the scope of the following claims.

What is claimed is:

1. A computer implemented method for optimizing energy transfer and conversion in quantum systems based on pre-defined criteria and for creating corresponding device designs, comprising:
(a) providing a database comprising input variables of one or more quantum systems in a lattice sample of a single material or an alloy or a composite material;
(b) modeling an initial crystal structure of the lattice sample at a first set of environmental parameters via a computing engine;
(c) adding a dopant to the lattice sample and determining a new equilibrium state of the lattice sample at a second set of environmental parameters via the computing engine;
(d) determining lattice-related oscillator characteristics in the new equilibrium state of the lattice sample via the computing engine, wherein the lattice-related oscillator characteristics comprise one of phonon-modes and/or photon absorption;
(e) estimating state transition rates for a first quantum system in the lattice sample in the absence of any strong coupling to a second quantum system via the computing engine;
(f) determining presence of any coupling and coupling strength of the first quantum system to the second quantum system via the computing engine;
(g) providing coherent stimulation of the lattice sample, thereby populating oscillator modes that the first quantum system and the second quantum system participate in;
(h) determining presence of coupling and enhancement of coupling strength of the first quantum system to the second quantum system after the coherent stimulation of the lattice sample via the computing engine;
(i) determining energy transfer and conversion dynamics as a result of the coherent stimulation of the lattice sample and the enhanced coupling of the first quantum system to the second quantum system via the computing engine;
(j) determining output variables of the energy transfer and conversion dynamics between the first quantum system and the second quantum system in the lattice sample via the computing engine.

2. The method of claim 1, wherein the input variables comprise compositions and structure of the single material or alloy or composite material of the lattice sample, energy levels, state lifetimes, and multipolarity of the quantum systems in the lattice sample, geometric arrangements of the quantum systems in the lattice sample, and characteristics of the coherent stimulation of the quantum systems in the lattice sample.

3. The method of claim 2, wherein the coherent stimulation is carried out by a laser and the characteristic of the coherent stimulation comprise a laser wavelength λₚ, pulse energy Eₚ, pulse length tₚ, repetition rate rₚ, and spot size Aₚ.

4. The method of claim 1, wherein each set of environmental parameters comprise a temperature, a pressure, an equilibrium time and applied electromagnetic field.

5. The method of claim 1, further comprising modelling formation and diffusion of dopant-stabilized vacancies in the new equilibrium state of the lattice sample at a third set of environmental parameters via the computing engine, after the addition of the dopant to the lattice sample.

6. The method of claim 1, wherein the output variables comprise form of energy, and amount of energy released from the lattice sample as result of the coherent stimulation applied to the lattice sample, overall energy balance ΔE, list of reaction products and particles and transition rates.

7. The method of claim 1, further comprising substituting the dopant with a different dopant or adding a different dopant and reiterating steps (c) to (j) via the computing engine.

8. The method of claim 1, wherein the quantum systems comprise one or more of nuclei, atoms, ions, and molecules.

9. The method of claim 1, wherein the equilibrium state of the lattice sample and phase stabilities are calculated with a DFT-based method comprising one of Quantum Espresso software package, Atomic Simulation Environment (ASE) software package, VASP with Phonopy software packages, or an open-source stochastic self-consistent harmonic approximation (SSCHA) software package.

10. The method of claim 1, further comprising calculating background electron density in the vicinity of the dopant via a DFT-based method comprising one of Quantum Espresso, ASE, VASP with Phonopy software packages, or SSCHA software packages.

11. The method of claim 1, further comprising estimating background electron density in the vicinity of the dopant based on experimental and theoretical values given in the literature.

12. The method of claim 1 wherein the energy transfer under step (i) can be accompanied by forms of energy conversion such as upconversion and downconversion if there is a suitable configuration of donor systems and receiver systems that enable such dynamics.

13. A non-transitory computer-readable storage medium containing a computer program for optimizing energy transfer and conversion in quantum systems, and for creating corresponding device designs, wherein the computer program when executed by a computing processor comprises:
(b) accessing a database comprising input variables of one or more quantum systems in a lattice sample of a single material or an alloy or a composite material;
(b) modeling an initial crystal structure of the lattice sample at a first set of environmental parameters;
(c) adding a dopant to the lattice sample and determining a new equilibrium state of the lattice sample at a second set of environmental parameters;
(d) determining lattice-related oscillator characteristics in the new equilibrium state of the lattice sample, wherein the lattice-related oscillator characteristics comprise one of phonon-modes and/or photon absorption;
(e) estimating state transition rates for a first quantum system in the lattice sample in the absence of any strong coupling to a second quantum system;
(f) determining presence of any coupling and coupling strength of the first quantum system to the second quantum system;
(g) providing coherent stimulation of the lattice sample, thereby populating oscillator modes that the first quantum system and the second quantum system participate in;
(h) determining presence of coupling and enhancement of coupling strength of the first quantum system to the second quantum system after the coherent stimulation of the lattice sample;
(i) determining energy transfer and conversion dynamics as a result of the coherent stimulation of the lattice sample and the enhanced coupling of the first quantum system to the second quantum system;
(j) determining output variables of the energy transfer and conversion dynamics between the first quantum system and the second quantum system in the lattice sample.

14. The non-transitory computer-readable storage medium of claim 13, wherein the input variables comprise compositions and structure of the single material or alloy or composite material of the lattice sample, energy levels, state lifetimes, and multipolarity of the quantum systems in the lattice sample, geometric arrangements of the quantum systems in the lattice sample, and characteristics of the coherent stimulation of the quantum systems in the lattice sample.

15. The non-transitory computer-readable storage medium of claim 14, wherein the coherent stimulation is carried out by a laser and the characteristic of the coherent stimulation comprise a laser wavelength λₚ, pulse energy Eₚ, pulse length tₚ, repetition rate rₚ, and spot size Aₚ.

16. The non-transitory computer-readable storage medium of claim 13, wherein each set of environmental parameters comprise a temperature, a pressure, an equilibrium time, and applied electromagnetic field.

17. The non-transitory computer-readable storage medium of claim 13, further comprising modelling formation and diffusion of dopant-stabilized vacancies in the new equilibrium state of the lattice sample at a third set of environmental parameters via the computing engine, after the addition of the dopant to the lattice sample.

18. The non-transitory computer-readable storage medium of claim 13, wherein the output variables comprise form of energy, and amount of energy released from the lattice sample as result of the coherent stimulation applied to the lattice sample, overall energy balance ΔE, list of reaction products and particles, and transition rates.

19. The non-transitory computer-readable storage medium of claim 13, further comprising substituting the dopant with a different dopant and reiterating steps (c) to (j) via the computing engine.

20. The non-transitory computer-readable storage medium of claim 13, wherein the quantum systems comprise one or more of nuclei, atoms, ions, and molecules.

21. The non-transitory computer-readable storage medium of claim 13, wherein the equilibrium state of the lattice sample and phase stabilities are calculated with a DFT- based method comprising one of Quantum Espresso software package, Atomic Simulation Environment (ASE) software package, VASP with Phonopy software packages, or an open-source stochastic self-consistent harmonic approximation (SSCHA) software package.

22. The non-transitory computer-readable storage medium of claim 13, further comprising calculating background electron density in the vicinity of the dopant via a DFT-based method comprising one of Quantum Espresso, ASE, VASP with Phonopy software packages, or SSCHA software packages.

23. The non-transitory computer-readable storage medium of claim 13, further comprising estimating background electron density in the vicinity of the dopant based on experimental and theoretical values given in the literature.

24. The non-transitory computer-readable storage medium of claim 13, wherein the energy transfer under step (i) can be accompanied by forms of energy conversion such as upconversion and downconversion if there is a suitable configuration of donor systems and receiver systems that enable such dynamics.
